(19) United States
(12) Patent Application Publication
(10) Pub. No.: US 2023/0298718 A1
(43) Pub. Date: Sep. 21, 2023

(54) METHODS AND SYSTEMS FOR REDUCING OPIOIDS FROM PAIN REGIMENS BY GRADUALLY REPLACING THE OPIOID DOSES WITH CANNABINOIDS

(71) Applicant: Green Sky Creations LLC, Seattle, WA (US)

(72) Inventors: Simon Robinson, Seattle, WA (US); Justin Esterberg, Seattle, WA (US); Brad Douglass, Seattle, WA (US)

(21) Appl. No.: 17/697,253

(22) Filed: Mar. 17, 2022

(51) Int. Cl. G16H 20/10

(52) U.S. Cl. CPC G16H 20/10

(57) ABSTRACT

A method for reducing opioids in a pain-modulating regimen of a patient includes generating a dosage scheme for the patient based on parameters associated with the patient's health condition. The dosage scheme includes a first dosage scheme for an opioid compound and a first dosage scheme for a cannabinoid compound to be administered to the patient concurrently. The first dosage scheme for the opioid compound includes a first plurality of dosages with a decreasing amount of an active ingredient in the opioid compound over a period of time. The method also includes determining an addiction risk score for the patient based on the dosage scheme and determining whether to adjust the dosage scheme based on the addiction risk score for the patient. In response to determining to adjust the dosage scheme, the method includes generating an adjusted dosage scheme based on the addiction risk score indicators.

### FIELD OF THE DISCLOSURE

The present disclosure is generally related to software kits for pharmaceutical treatments for pain and inflammation management. The present disclosure relates more specifically to software kits for aiding in administration of opioids, or other classes of drugs, and cannabinoids with a dosing schema to prevent or reverse dependency.

### BACKGROUND

Opioid addiction is a debilitating disease characterized by physical and psychological reliance on opioids, a class of molecules found in certain prescription pain medications and illegal drugs such as heroin. The United States is in the midst of an opioid abuse epidemic. Opioid addiction commonly starts in patients who are prescribed painkillers but fail to detox from opioids after an extended period of time due to the persistence of pain and/or increasing dependence on the prescription drugs containing opioids.

Over time, patients may become tolerant to opioids, which can lead to an escalation in opioid dosing. Escalated dosing can exacerbate withdrawal effects when opioids are removed and increase the risk of addiction. Long-term opioid use is associated with poor health outcomes including overdose, organ system failures, and death. Additionally, patients who are opioid-dependent preoperatively have been shown to have worse clinical outcomes following surgical treatment.

There is a need to provide a method for patients with acute and chronic pain to avoid unnecessarily high dosages of opioids or safely taper off, and eventually eliminate, the use of opioids to improve health outcomes. Many patients are turning to cannabis and/or extracts of cannabis, which contain pain-relieving cannabinoids, such as cannabidiol (CBD) and tetrahydrocannabinol (THC), as well as other non-cannabinoid compounds to treat pain. Areas of the United States with legal access to medical and adult-use cannabis products show a decrease in rates of opioid use. While physicians are largely prevented, for a host of reasons, from actively prescribing cannabinoids as a treatment for pain, this could change in the future, as medical science and regulatory regimes increasingly accept the validity of cannabis and cannabis-derived compounds for certain patients.

A software kit for reducing dependence on opioids may be useful for a wide variety of practitioners, patients, and opioid-dependents who wish to transition from opioid-based pain management to cannabinoid pain management, which may involve significantly less dependency, addiction potential, and overall health hazards. Such software kits could be provided in pharmaceutical settings where dosing can be tightly controlled and monitored and provide patients and those dependent on opioids with relief from symptoms associated with opioid withdrawal.

### DESCRIPTIONS OF THE DRAWINGS

FIG. 1 illustrates a system for eliminating opioids from a patient's pain regimen by gradually replacing the opioid doses with cannabinoids and other pain treatments, in accordance with some embodiments.

FIG. 2 illustrates a software kit database, in accordance with some embodiments.

FIG. 3 illustrates a dosage module, in accordance with some embodiments.

FIG. 4 illustrates a user database, in accordance with some embodiments.

FIG. 5 illustrates a user device interface module, in accordance with some embodiments.

FIG. 6 illustrates a flowchart showing a method for training a machine learning model, in accordance with some embodiments.

FIG. 7 is a block diagram of a computer system as may be used to implement features of some embodiments of the disclosed technology.

FIG. 8 is a block diagram illustrating an overview of a system on which some implementations of the disclosed technology can operate, in accordance with some embodiments.

### DETAILED DESCRIPTION

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

The devices, systems, and methods of the present disclosure provide for an improved, automatically adjustable way of reducing opioids in a patient's pain-modulating regimen. The present disclosure reduces the risk of opioid addiction leading to long-term use of opioids. Long-term use of opioids may lead to poor health, organ system failures, or even death from overdose. The method is based on generating a dosage scheme for a patient based on parameters associated with the patient's health condition. The dosage scheme includes dosage schemes for an opioid compound and a cannabinoid compound so that the dosages of opioids decrease over a period of time. The dosage scheme can then be automatically adjusted based on the pain level of the patient. The patient's pain level is assessed based on feedback received (e.g., on a standardized pain scale) from the patient or based on physiological pain level indicators based on biometric data. Examples of commonly used opioid drugs include but are not limited to oxycodone, codeine, hydrocodone, hydromorphone, oxymorphone, methadone, morphine, fentanyl and tapentadol.

In accordance with some embodiments, a method for reducing opioids in a pain-modulating regimen of a patient is performed by a computer system. The method includes generating, by a computer system, a dosage scheme for the patient based on parameters associated with the health condition of the patient. The dosage scheme includes a first dosage scheme for an opioid compound and a first dosage scheme for a cannabinoid compound. The opioid and the cannabinoid are to be administered to the patient concurrently over a period of time. The first dosage scheme for the opioid compound includes a first plurality of dosages with a decreasing amount of an active ingredient in the opioid compound over the period of time. The method also includes receiving pain level indicators associated with the patient. The pain level indicators include a first pain level indicator based on an input from the patient and a second pain level indicator based on biometric data obtained by a wearable device. The wearable device is configured to be worn by the patient. The method includes determining, by the computer system, whether to adjust the dosage scheme based on the pain level indicators. In response to determining to adjust the dosage scheme, the method includes generating an adjusted dosage scheme based on the pain level indicators. The adjusted dosage scheme includes a second dosage scheme for the opioid compound. The adjusted dosage scheme is different from the first dosage scheme for the opioid compound to be administered to the patient.

In accordance with some embodiments, a method for administering drugs to a patient includes generating, by a computer system, an initial dosage scheme for the patient based on parameters associated with a health condition of the patient. The initial dosage scheme includes a first dosage scheme for a first drug and a first dosage scheme for a second drug to be administered to the patient concurrently over a period of time. The first dosage scheme for the first drug includes a first plurality of dosages with a decreasing amount of an active ingredient in the first drug over the period of time. The method includes receiving, by the computer system, one or more health condition indicators associated with the patient. The one or more health condition indicators include an indicator based on an input from the patient. The method also includes generating, by the computer system, a subsequent dosage scheme based on the one or more health condition indicators.

In accordance with some embodiments, a method for opioid management in a pain-modulating regimen for a patient includes receiving, by a computing system, patient data of a patient. The method includes generating, by at least one trained cannabinoid dosing machine learning model, a pain relief regimen based on the received patient data. The pain relief regimen includes an opioid tapering dosage scheme and a cannabinoid dosage scheme. The cannabinoid is designed to compensate for opioid tapering of the opioid tapering dosage scheme such that the pain relief regimen effectively alleviates the pain of the patient. In some embodiments, the present disclosure includes a semi-autonomous system for mitigating the risks of opioids with cannabinoids. The system may taper the quantity of opioids to minimize the risk of opioid dependence. For example, the system provides for dosage schemes of reducing an administered dosage of opioids while concurrently administering cannabinoids to manage a patient's pain. The risk associated with opioids may be physical dependence or addiction. In some embodiments, the risk associated with opioids is respiratory arrest or death, or liability on the part of the prescribing physician for over-prescribing or mis-prescribing. In some embodiments, cannabinoids may be included in the dosage scheme irrespective of non-medical prescribing limitations.

In some embodiments, the system interfaces with opioid-prescribing monitoring databases to maintain compliance.

In some embodiments, biometric data, as well as input provided by a patient (e.g., a user), are analyzed to adjust dosage regimens in real time. The system also may optimize the dosage scheme toward the minimal quantity of necessary opioids over time.

In some embodiments, the system provides indications for dosages including one or more compounds possessing phytocannabinoid-based chemical structures or exhibiting pharmacological activity on the endocannabinoid system. The cannabinoid compound may be selected from a group consisting of tetrahydrocannabinolic acid (THCA), tetrahydrocannabinol (THC), cannabidiolic acid (CBDA), cannabidiol (CBD), cannabigerolic acid (CBGA), cannabigerol (CBG), cannabichromenic acid (CBCA), cannabichromene (CBC), cannabinol (CBN), cannabielsoin (CBE), cannabicyclol (CBL), and cannabicitran (CBT), in addition to all respective isomers and human metabolites of in-group molecules. “A cannabis compound” shall mean any constituent extracted or derived from a plant belonging to the genus Cannabis, including, but not limited to cannabinoid, terpenoid and flavonoid compounds as well as synthetic, semisynthetic or highly purified versions of any such constituent.

In some embodiments, the system is for mitigating the risks of anesthetics and sedatives with cannabinoids. The system may successfully taper the quantity of sedatives or anesthetics to minimize the risk of the patient regaining consciousness unexpectedly. The risk associated with sedatives or anesthetics may be respiratory arrest or death. In some embodiments, the risk associated with anesthetics is physical dependence developed by the anesthesiologist due to environmental exposure to unnecessary anesthetics.

In some embodiments, the cannabinoids are administered via the same or different route of administration as sedatives or anesthetics.

In some embodiments, the system is for decreasing or optimizing the total consumption of prescription drugs.

A Software Kit and Processes for Reducing Opioids from Patient's Pain-Modulating Regimen

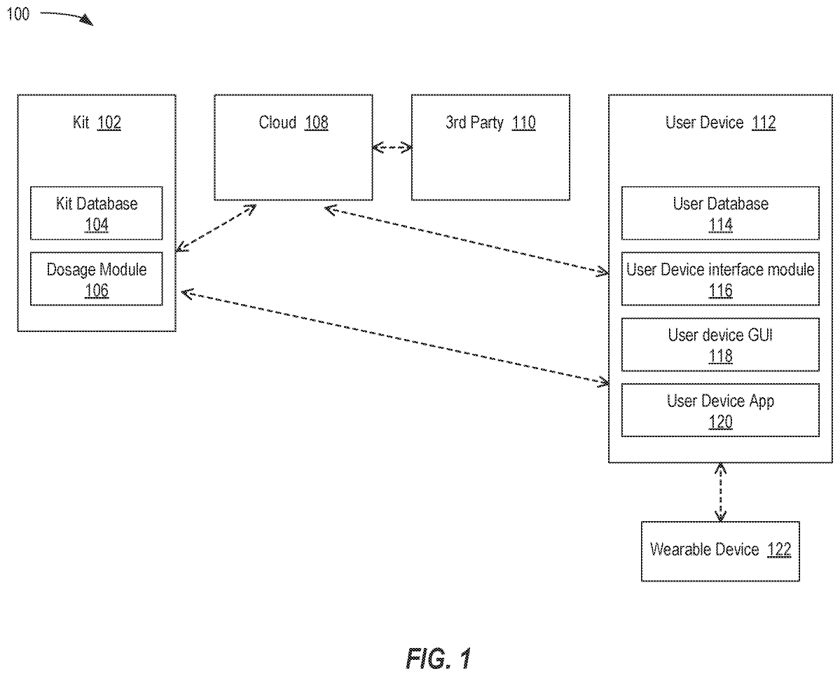

FIG. 1 shows a system 100 including a software kit 102 (e.g., a software application or a program) for eliminating opioids from a patient's pain regimen by gradually replacing the opioid doses with cannabinoids and other less harmful pain treatments. The other pain treatments may include acetaminophen, non-steroidal anti-inflammatory drugs (e.g., aspirin, ibuprofen, naproxen), and other drugs; plant-derived extracts and complex mixtures (e.g., kratom, turmeric, white willow, and Boswellia); and mind-body techniques (e.g., acupuncture and meditation). The software kit 102 offers a solution for pain management and opioid cessation in patients who have been prescribed painkillers but fail to detox from opioids after an extended time due to the persistence of pain and/or increasing dependence on the prescription drugs. The software kit 102 may help mitigate the risk of a patient becoming tolerant to opioids, which often leads to an escalation in dosing of opioids—a strong risk factor for addiction. The system 100 further includes a user device 112 that is in communication with the software kit 102 and a cloud 108 (e.g., a cloud server) for facilitating the communications and operations of the system 100. In some embodiments, the system 100 further includes a third-party device 110.

The software kit 102 may offer a solution for patients with acute and chronic pain by eliminating the use of opioids and improving health outcomes through the intelligent use of cannabis, cannabinoids (e.g., THC, CBD, and chemical/physiological analogues), and/or extracts of cannabis to treat pain. Thus, the software kit 102 may determine a dosing scheme based on pain severity and expected length, i.e., addition of the THC, CBD, and/or analogues in a specific ratio for a specific pain level such that higher pain levels may be associated with higher doses of the dosing scheme or higher ratios of specific components within the scheme.

The software kit 102 may include a software kit database 104 having indications of the pharmaceutical doses of the drugs measured out in relevant periods such as hours in a day, days, weeks, months, etc. Indications of pharmaceutical doses refer to information or data associated that can be provided to the patient (e.g., by displaying the indication on a display of a computer device). The indications of the pharmaceutical doses can include volumes, weights, concentrations, dosing frequency, pharmaceutical identifiers, etc., associated with the pharmaceutical compounds. The software kit 102 may have a pharmaceutical composition including a cannabinoid and a nonsteroidal anti-inflammatory drug (NSAID).

The pharmaceutical doses may be in any one or a combination of forms, such as pills, capsules, tablets, transdermal patches, injections, tinctures, smokable herbs, vaporizers, or any other form in which opioids or cannabinoids may be administered. The nonsteroidal anti-inflammatory drug (NSAID) may be, for example, aspirin, ibuprofen, naproxen, etc. The pharmaceutical composition has a cannabinoid composition, which includes a cannabis compound, a pain management composition, which may be pharmaceutical and/or botanical, and an opioid cessation composition (e.g., methadone). In one embodiment, the software kit 102 may offer opioid cessation including compositions of opioids and cannabinoids in various proportions. The proportion of opioids may gradually decrease throughout the software kit 102 compositions as the proportions of cannabinoids gradually increase. It can be noted that the cannabinoids may be selected for pain management therapy. The final composition of the software kit 102 may contain zero or a negligible quantities of opioids. In addition, the composition of cannabinoids can also follow a tapering regimen after the conclusion of opioids.

In some embodiments, the software kit 102 may offer matching of cannabinoids and cannabis compounds with nutritional supplements for synergistic effects. The software kit 102 may facilitate assessing the intended effect of a nutritional supplement and matching it with the cannabinoid extract with the most synergistic properties.

In some embodiments, the software kit 102 may be coupled to the user device 112 to facilitate user feedback to the software kit 102, via a user device app 120 on the user device 112. The user feedback may allow the regimen to be adjusted based on a change in a patient's pain level. For example, improvements in pain can produce a new regimen with reduced dosing in real time such as advising the patient to halve the next suggested dose or skip a dose entirely. The patient feedback can include pain scores on a standardized pain score scale. Such standardized pain score scale can include visual analog scale (VAS), numerical rating scale (NRS), defense and veterans pain rating scale (DVPRS), non-verbal pain scale (NVPS), pain assessment in advanced dementia scale (PAINAD), behavioral pain scale (BPS), critical-care pain observation tool (CPOT), or any other standardized pain scale score.

The user device 112 may also help in tracking the usage of the software kit 102 over time as well as continually monitoring the patient's pain. Tracking of the usage of the software kit 102 and the patient's pain may be achieved by making a graph in which the Y-axis displays dosing measures of opioid (e.g., oxycodone) pain management and the X-axis displays dosing measures of the cannabinoids. It should be noted that the chart may have a negative or downward (left-to-right) slope since the opioid doses decrease as the cannabinoid doses increase, creating an inverse relationship, and each point on the chart could represent one week, day, or hour in the pain regimen.

In one embodiment, the software kit 102 may include specific instructions for the dosing regimen and for the need to wean patients off opioids (based on risks and dangers of dependency) as well as information about the effectiveness and safety of the cannabinoid formulation. In one case, a plurality of software kits 102 might be available and the physician may prescribe a particular software kit 102 to the patient based on the pain level, tolerance, doses, etc., of the patient. In another case, the software kit 102 may be customized for each patient by the physician based on the pain level, tolerance, doses, etc., of the patient. In one embodiments, the software kit 102 may be chosen based on the duration of post-operation (e.g., surgical) care, with dosing for specific pain types and magnitudes. In some embodiments, the software kit 102 may be chosen based on a need to reduce opioids pre-operatively. It can be noted that once the pain is at a manageable level, the patient may be allowed to reorder cannabinoid therapy without a prescription, but would perhaps need to go to the doctor and have pain assessed for additional opioid doses.

For example, a topical CBD can prevent pain and modulate inflammation in some pseudo-surgical applications (for example, applying topical CBD prior to a tattooing operation). Thus, topically applied presurgical and post-surgical CBD could be an effective pain and inflammation management tool. In one embodiment, one or more software kits 102 may be available and choices for the software kit 102 may be based on the type of injury/surgery/operation. For example, a patient experiencing liver failure may opt to receive a liver transplant and the recovery from such a transplant may take 2 to 4 months. The patient may have an initial pain intensity of 8 that may gradually decrease with time and may require 100 mg of an opioid, 100 mg of a cannabinoid, and a total of 1800 mg of therapeutic agents of all types on the day of surgery. The amount of opioid(s) and cannabinoid(s) may change based on the change in pain intensity. Hence, as the pain decreases in magnitude the quantity of opioid(s) administered may decrease while the quantity of cannabinoid(s) may increase.

In one embodiment, the software kit 102 may be associated with a software app, which tells the patient which elements of the software kit 102 to use, based on the patient pain check-in or a questionnaire. The software kit database 104 of the software kit 102 may include pharmaceutical doses of the drugs measured out in relevant periods such as hours of the day, days, weeks, months, etc. The software kit database 104 may also include a combination of opioid(s), cannabinoid(s), and other painkillers in various proportions based on user parameters.

The pharmaceutical doses may be in any one or a combination of forms, such as pills, capsules, tablets, transdermal patches, injections, tinctures, smokable herbs, vaporizers, or any other form in which opioids or cannabinoids may be administered. In one embodiment, the software kit database 104 may also include dosages of drugs before surgery to wean patients onto cannabinoid treatment after surgery since administering cannabinoids before pain actually occurs can decrease both the perception and the physiological manifestation of pain. Reduction of opioids preoperatively may lead to a better control of a patient's pain during and after surgery thereby causing an improved outcome for the patient. For example, the software kit database 104 includes, for two days before surgery, a dosage of 25 mg of 11-OH delta-9 tetrahydrocannabinol (11-OH-d9-THC) for the patient experiencing the liver failure, twice a day. The software kit database 104 may include indications of pharmaceutical doses of drugs measured out in relevant hours of day and days for short-term pain. For example, the software kit database 104 indicates that, for the day of surgery, a dosage of 25 mg of hydrocodone and 25 mg of CBD is to be administered to the patient 4 times a day, and 500 mg of acetaminophen and 300 mg of ibuprofen are to be administered twice a day. The software kit database 104 may include indications of pharmaceutical doses of drugs measured out in week- and month-long periods for pain of extended duration. For example, the software kit database 104 includes, for the first week post-surgery, a regimen of 25 mg of hydrocodone, 25 mg of CBD, 500 mg of acetaminophen, and 300 mg of ibuprofen twice a day.

In some embodiments, the software kit 102 includes a dosage module 106 that may receive data from the patient via a user device interface module 116. In some embodiments, the system 100 includes interfaces between dosage module 106 and user device interface module 116 and prescription monitoring databases to ensure that both the prescriber and the patient maintain compliance with all local, state, and federal statutes and codes. Such background interfacing between the semi-autonomous prescribing system and the prescription monitoring database could serve as a checksum to protect the health of the patient and the potential liability of the prescriber. In one embodiment, the data may include a pain level received from the patient through the user device interface module 116. For example, the dosage module 106 receives the pain level of 8 (out of 10) associated with the patient with the liver failure on the day of his surgery, from the user device interface module 116. In some embodiments, the dosage module 106 may retrieve the user parameters from a user database 114. For example, the dosage module 106 extracts the user parameters, i.e., gender, age, weight, height, medical conditions, personality, and disposition associated with the patient from the user database 114. The medical conditions may include medical conditions that are associated with pain or that can cause pain. The medical conditions may further include any medical conditions that may affect the effectiveness of any pain relief medications. The medical conditions may also include conditions that can be affected by any pain relief medications. The disposition may refer to a disposition to addiction including a history of addictive behavior, genetic disposition to addiction or drug or alcohol abuse, et cetera.

In some embodiments, the dosage module 106 matches the pain level and the user parameters with a dose level. In some embodiments, the dose level may be retrieved from the software kit database 104. For example, the dosage module 106 matches the pain level of 8 (out of 10) associated with the patient and the user parameters associated with the patient with the dose level on the day of the patient's surgery. For example, the patient's pain level and parameters are matched with a dose level including 25 mg of hydrocodone and 25 mg of CBD 4 times a day, and 500 mg of acetaminophen and 300 mg of ibuprofen twice a day, i.e., ((25×4)+(25×4)+(500×2)+(300×2)) mg=1800 mg of total medications or medicaments per day. The dosage module 106 may further determine the dosage amount for the patient. For example, the dosage module 106 determines that the patient requires 25 mg of hydrocodone and 25 mg of CBD 4 times a day, and 500 mg of acetaminophen and 300 mg of ibuprofen twice a day, i.e., ((25×4)+(25×4)+(500×2)+(300×2)) mg=1800 mg of total painkiller and inflammation-modulating compounds on the day of his surgery. In some embodiments, the dosage amount is determined based on the software kit database 104. In some embodiments, the dosage module 106 may determine which prescription is required for the patient. The prescription may be dependent on the day associated with the surgery (such as the day of surgery or 2 days after surgery).

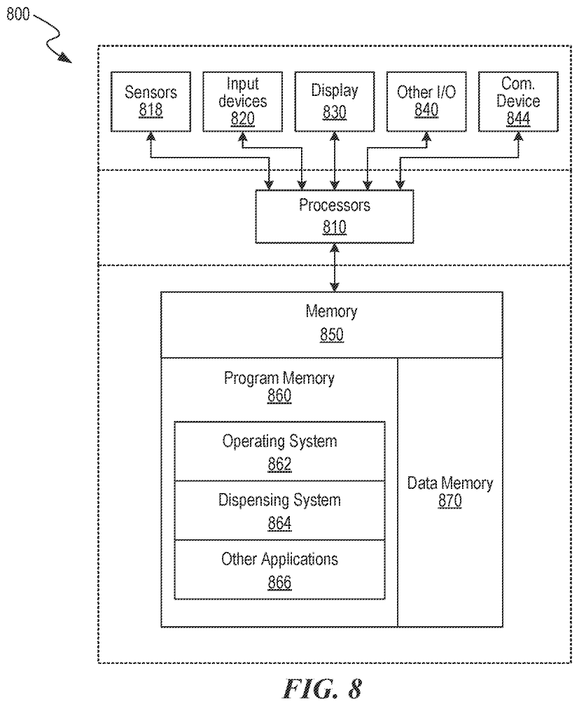

In one embodiment, the dosage module 106 may determine the dosage amounts for the patient for a week with a decreasing quantity of opioids and an increasing quantity of cannabinoid(s). For example, 1st day—1800 mg, 2nd day—1800 mg, 3rd day—1800 mg, 4th day—1800 mg, 5th day—1810 mg, 6th day—1820 mg, and 7th day—1810 mg. In another embodiment, the dosage module 106 may determine the dosage amounts for the patient for one or more weeks with a decreasing quantity of opioids and an increasing quantity of cannabinoid(s). For example, 1st week—1800 mg, 2nd week—1875 mg, 3rd week—2850 mg, 4th week—3025 mg, 5th week—3220 mg, 6th week—4210 mg, 7th week—4405 mg, and >7th week—4400 mg. The dosage module 106 may extract the dosage indications from the software kit database 104. For example, the dosage module 106 extracts from the software kit database 104 that the patient requires 1800 mg of painkillers on the day of his surgery. Based on the extracted dosages from the software kit database 104, the dosage module 106 may send the dosage notification to the user device interface module 116. For example, the dosage module 106 sends a notification to the user device interface module 116 stating that the patient requires 1800 mg of painkillers on the day of his surgery, i.e., 25 mg of hydrocodone and 25 mg of CBD 4 times a day, and 500 mg of acetaminophen and 300 mg of ibuprofen twice a day. In one embodiment, the dosage module 106 may also send risks of dependence and alternative therapies to the user device interface module 116. In some embodiments, the dosage module 106 may utilize the concept of artificial intelligence (AI) to determine the dosages as well as risks of dependence and alternative therapies. The processes for training and applying artificial intelligence to determine the dosages, as well as risks, are discussed in detail with respect to FIG. 8.

The system 100 may also include the cloud 108. The cloud 108 or communication network may be a wired and/or a wireless network. The communication network, if wireless, may be implemented using communication techniques such as Visible Light Communication (VLC), Worldwide Interoperability for Microwave Access (WiMAX), Long Term Evolution (LTE), Wireless Local Area Network (WLAN), infrared (IR) communication, Public Switched Telephone Network (PSTN), radio waves, and other communication techniques known in the art. The communication network may allow ubiquitous access to shared pools of configurable system resources and higher-level services that can be rapidly provisioned with minimal management effort, often over the internet, and relies on sharing of resources to achieve coherence and economies of scale, like a public utility, while third-party clouds enable organizations to focus on their core businesses instead of expending resources on computer infrastructure and maintenance.

In some embodiments, the system 100 may include a third-party device 110 (e.g., a computer device such as a laptop, smartphone, tablet, computer, smart speaker, or input/output (I/O) device) such as, but not limited to, a device associated with a doctor, caretaker, physician, or pharmacy, etc. The third-party device 110 may add new data or update existing data in the software kit database 104 based on the user parameters such as pain intensity, gender, age, weight, height, medical conditions, personality, and disposition. The third-party device 110 may add indications of pharmaceutical doses of drugs measured out in relevant hours of day and days for short-term pain. The third-party device 110 may be used to prescribe different types of cannabinoid treatment for individual patients. For example, some patients may end up with some level of chronic pain and the third-party device 110 may be used to prescribe dosages for treatment of the chronic pain. As another example, the third-party device 110 adds, for the day of surgery, a dosage of 25 mg of hydrocodone and 25 mg of CBD for the patient 4 times a day, and 500 mg of acetaminophen and 300 mg of ibuprofen twice a day. The third-party device 110 may add pharmaceutical doses of drugs measured out in week and month for long-term pain. For example, the third-party device 110 adds, for the first week post-surgery, a dosage of 25 mg of hydrocodone and 25 mg of CBD for the patient four times a day, and 500 mg of acetaminophen and 300 mg of ibuprofen twice a day.

In some embodiments, the system 100 may include one or more user devices 112 (e.g., a computer device such as a laptop, smartphone, tablet, computer, smart speaker, or I/O device). In some embodiments, the user device 112 corresponds to, includes, or is in communication with a wearable device 122. In some embodiments, the wearable device 122 is a smartwatch, a wristband, a fitness tracker, or a wearable medical device. The wearable device 122 includes one or more sensors configured to detect one or more health condition indicators associated with the patient. The health condition indicators include one or more of a pain level, a heart rate, a temperature, a blood pressure, a respiration rate, an oxygenation level, a motor activity level, a pupil constriction/dilation level, and sleep phase information. The wearable device 122 may utilize the user device 112 as additional memory or computing power or connection to the internet.

In some embodiments, feedback on efficacy (patient and/or caretaker feedback plus biometric data) can be collected from user device 112 via input by humans or via interfacing with external biometric sensors (e.g., the heart rate, temperature, respiration rate, oxygenation levels, motor activity levels, pupil constriction/dilation, sleep phases, etc.). A key element of such a software kit 102 enabled with user and biometric data input is the ability to adjust the treatment algorithm and/or dosing schedule automatically and in real time. In some embodiments, the software kit 102 may utilize a user database 114 that may contain the data received by the user device interface module 116. The user database 114 may contain the basic user data such as gender, age, weight, height, medical conditions, personality, and disposition. In addition, the user database 114 can contain active-pharmaceutical ingredient-specific data that may also be relevant in tailoring dosage regimens of pharmacologically active compositions. For example, tailoring may include collecting and querying information about the patient's opioid history prior to prescription, patient-reported outcomes, side effects previously reported, and efficacy of prior prescription regimens. The patient-reported outcomes may include pain scores on the standardized pain score scale such as visual analog scale (VAS), numerical rating scale (NRS), defense and veterans pain rating scale (DVPRS), non-verbal pain scale (NVPS), pain assessment in advanced dementia scale (PAINAD), behavioral pain scale (BPS), critical-care pain observation tool (CPOT), or any other standardized pain scale score.

In some embodiments, the user device interface module 116 may be triggered when the patient logs in to the user device app 120, on the user device 112. The user device interface module 116 may enable the patient to extract dosages associated with a particular pain regimen of the patient. The user device interface module 116 may continuously monitor for a dosage notification from the dosage module 106. The user device interface module 116 may receive the dosage notification from the dosage module 106. For example, the user device interface module 116 receives notification from the dosage module 106 that the patient requires 1800 mg of painkillers on the day of his surgery, i.e., 25 mg of hydrocodone and 25 mg of CBD 4 times a day, and 500 mg of acetaminophen and 300 mg of ibuprofen twice a day. The user device interface module 116 may facilitate displaying the dosage notification on a graphical user interface (GUI) 118 of the user device 112. For example, the user device interface module 116 facilitates the display on the GUI 118 of an indication that the patient requires 1800 mg of painkillers on the day of his surgery, i.e., 25 mg of hydrocodone and 25 mg of CBD 4 times a day, and 500 mg of acetaminophen and 300 mg of ibuprofen twice a day. In one embodiment, the user device interface module 116 may also display risks of dependence and alternative therapies on the user device GUI 118. In some embodiments, the user device interface module 116 may utilize artificial intelligence (AI) to determine the risk of adverse events, including dependence, along with alternative therapies using both supervised and unsupervised training approaches.

In some embodiments, the system 100 includes, or is in communication with, a drug administration system or device. The system 100 may provide instructions to the drug administration system or device that indicate dosages of drugs to be administered to the patient. The drug administration system or device can be configured to control the quantity, volume, concentration, etc., of drugs administered to the patient.

In some embodiments, a drug administration device is configured to provide the patient with access to a particular dosage of tablets held in compartments. In some embodiments, the risk of addiction to opioids may be reduced by limiting the number of tablets available, allowing certain compartments to unlock at certain times of the day, on certain days, or on any schedule with medical utility. In one embodiment, a caregiver may need to unlock the compartments for the patient. In another embodiment, the dosage of drugs may be shipped or delivered to patients at appropriate times.

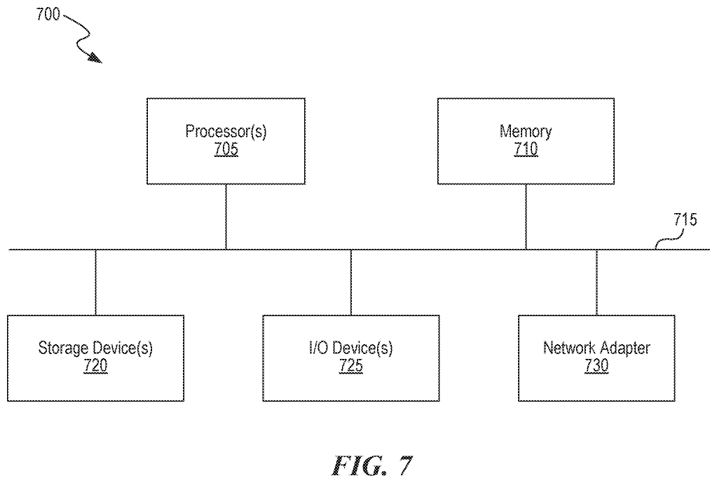

In some embodiments, the user device GUI 118 may include one or more affordances that may either accept inputs from patients, facilitate displaying outputs to patients, or perform both actions. The patient can interact with the user device GUI 118 via one or more input devices (e.g., input/output device 725 described with respect to FIG. 7). The input devices may include user input buttons, switches, knobs, levers, keys, trackballs, touchpads, cameras, microphones, motion sensors, heat sensors, inertial sensors, touch sensors, or a combination of the above. Additionally or alternatively to the user device GUI 118, a user interface may be implemented as a command-line interface (CLI), a voice interface, or a web-based user interface.

In some embodiments, the user device GUI 118 may enable the patient to input data related to calendars or calendar events. In one embodiment, the user device GUI 118 may send notifications in a user-friendly or interactive form to the patient. In some embodiments, the user device 112 includes a user device app 120 running on the user device 112. In one case, the user device app 120 is a mobile application. In another case, the user device app 120 is a web application. In some embodiments, the user device GUI 118 is associated with the user device app 120 and is configured to facilitate various operations such as, but not limited to, displaying user profile, tracking the usage of the apparatus over time, logging the dose taken by the patient, tracking the dose regimen of the patient, and enabling the patient to communicate with their physician or health care provider. In one embodiment, the user device app 120 may enable the patient to track the dosing. For example, the user device app 120 may provide a user device GUI 118 that shows the total dose provided to the patient, the maximum dose for a particular day, and the dose regimen over previous days. In one embodiment, the user device app 120 may enable the patient to enter subjective measures that relate to the dosing, such as pain levels, intoxication levels, etc. In some embodiments, the patient may request the caregiver (e.g., a caregiver associated with the third-party device 110) to modify the daily dose based on the subjective information. For example, the patient requests the caregiver to increase the dose of cannabinoids based on the increased pain level. In another embodiment, the patient may modify their own dose.

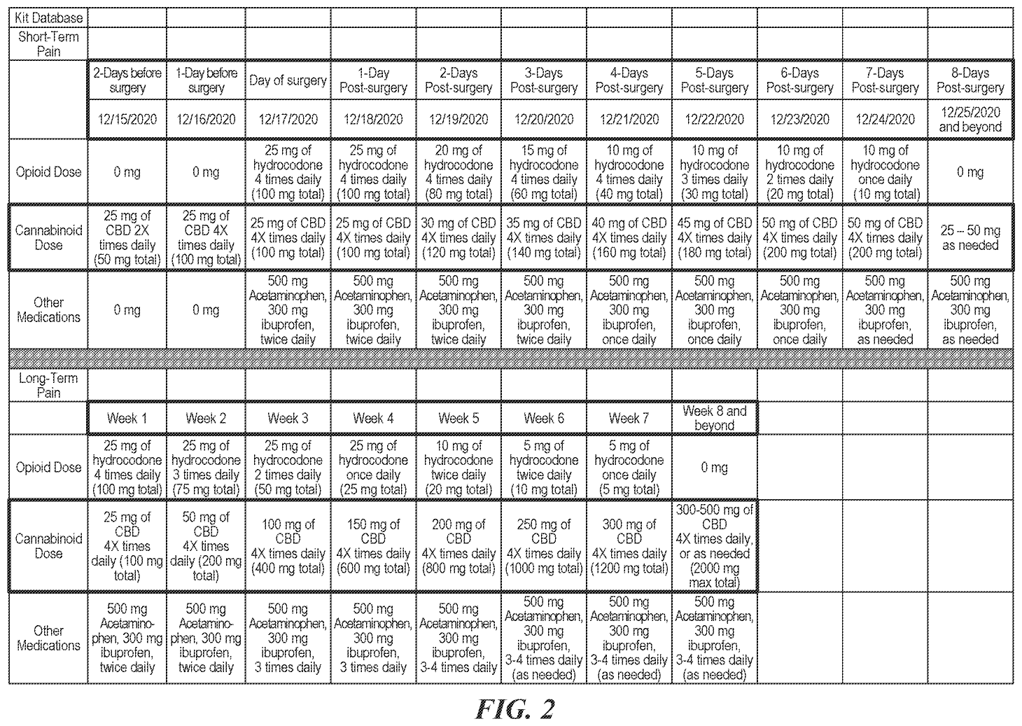

The functioning of the software kit database 104 will now be explained with reference to FIG. 2. The figure (i.e. FIG. 2) shows a screenshot of a software kit database 104 displayed by a computer device. The software kit database 104 includes multiple indications of pharmaceutical doses of the drugs measured out in relevant periods such as hours of the day, days, weeks, months, etc. The multiple indications of pharmaceutical doses are part of a dosage scheme for a period of time. For example, the upper rows correspond to a dosage scheme for an 11-day time period and the lower rows correspond to a dosage scheme for an 8-week period of time. The software kit database 104 includes a combination of opioids, cannabinoids (e.g., CBD), and other pain killers and/or inflammation-modulating substances in various proportions based on user parameters. The pharmaceutical doses may be in any one or a combination of forms, such as pills, capsules, tablets, transdermal patches, injections, tinctures, smokable herbs, vaporizers, or any other form in which opioids or cannabinoids may be administered. In one embodiment, the software kit database 104 may also include indications of doses of drugs before surgery to build up cannabinoids in a patient to reduce pain immediately after surgery, since, in some embodiments, administering cannabinoids before the occurrence of pain or injury (e.g., surgical incisions) can decrease the perception of pain and the actual levels of inflammation.

For example, in FIG. 2 the software kit database 104 includes, for 2 days before surgery, a dosage of 25 mg of CBD for the patient, twice a day. The software kit database 104 may include indications of pharmaceutical doses of drugs measured out in relevant hours of day and days for short-term pain and inflammation modulation. For example, the software kit database 104 includes, for the day of surgery, a dosage of 25 mg of hydrocodone and 25 mg of CBD for the patient 4 times a day, and 500 mg of acetaminophen and 300 mg of ibuprofen twice a day. The software kit database 104 may include pharmaceutical doses of drugs measured out in week and month for long-term pain. For example, the software kit database 104 includes, for the first week post-surgery, a dosage of 25 mg of hydrocodone and 25 mg of CBD for the patient four times a day, and 500 mg of acetaminophen and 300 mg of ibuprofen twice a day. Importantly, the software kit database 104 provides a dosing schema that continuously decreases the level of opioids administered in direct conjunction with the levels of pain and inflammation experienced by the patient such that at the conclusion of the dosing schema, the patient's pain management regimen may include only cannabinoids and/or NSAID pain treatment but zero opioids. In this way, the patient's opioid-based treatments are gradually reduced until completely eliminated.

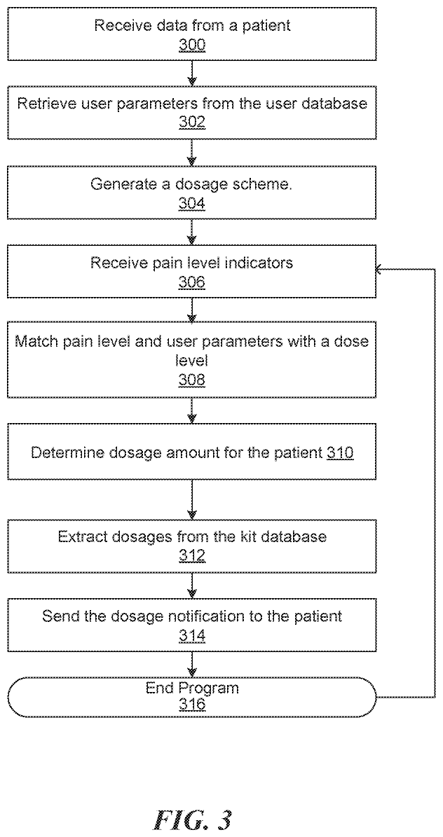

FIG. 3 is a flowchart illustrating processes performed by the dosage module 106 of the software kit 102. At step 300, the dosage module 106 may receive data from the patient via a user device interface module 116. In one embodiment, the data may be, but is not limited to, a pain level. For example, the dosage module 106 receives from the user device interface module 116 the pain level of 8 out of 10 (e.g., on a standardized pain scale) associated with the patient on the day of his surgery. The dosage module 106 may retrieve, at step 302, the user parameters from the user database 114 associated with the patient. In one embodiment, the user parameters may be, but are not limited to, gender, age, weight, height, medical conditions, and disposition. The medical conditions may include medical conditions that are associated with pain or that can cause pain. The medical conditions may further include any medical conditions that may affect the effectiveness of any pain relief medications. The medical conditions may also include conditions affected by any pain relief medications. The medical conditions can be long-term or short-term. For example, the medical conditions may describe a surgery that is expected to cause pain to the patient.

In another embodiment, the user parameters may be biometric information produced by sensors worn, carried, implanted into, or in the general vicinity of the patient that allow the monitoring of physiological parameters of the patient's corporeal body. In some embodiments, the user parameters are obtained by the wearable device 122 described above with respect to FIG. 1. For example, the dosage module 106 retrieves the user parameters such as gender—male, age—32, weight—110 kgs, height—6′2″, medical condition—diabetic, personality—strong, and disposition —4, associated with the patient from the user database 114 and biometric data such as heart rate—60 beats per minute, respiration rate—15 breaths per minute, oxygenation levels—90%, blood—oxygen saturation (% SpO₂ or SaO₂), motor activity levels—low, pupil size—constricted, sleep profile—low REM, etc.

At step 304, the dosage module 106 generates a dosage scheme. The dosage scheme provides for a plan of administering opioid compounds concurrently with other pain relief medications to the patient in accordance with the patient's pain level. The dosage scheme can be adjusted based on changing pain level. For example, a first dosage scheme is associated with a first pain level, a second dosage scheme is associated with a second pain level, and a third dosage scheme is associated with a third pain level.

In some embodiments, the dosage scheme includes a scheme for administering opioid compounds to the patient concurrently with other compensating pain relief medications. The other pain relief medications may include one or more of cannabinoid compounds, NSAID compounds, and other pain relief medications. In particular, the dosage scheme includes an opioid tapering dosage scheme including multiple dosages of opioids with decreasing active ingredient amounts over a period of time. The opioid tapering dosage scheme is configured to gradually reduce the total quantity of opioids in the patient's pain-modulating regimen. To compensate for the opioid tapering, the dosage scheme further includes a cannabinoid and/or other pain relief medication dosage scheme so that an overall effectiveness of alleviating the pain of the patient remains desirable. In some implementations, the dosage scheme is generated based on a trained cannabinoid dosing machine learning model described in further detail with respect to FIG. 8.

At step 306, the dosage module 106 receives pain level indicators associated with the patient. The pain level indicators may include a pain level score received from the patient and/or biometric data obtained from the patient. The pain level score may correspond to a pain level score provided based on the standardized pain score scale such as the visual analog scale (VAS), numerical rating scale (NRS), defense and veterans pain rating scale (DVPRS), non-verbal pain scale (NVPS), pain assessment in advanced dementia scale (PAINAD), behavioral pain scale (BPS), or critical-care pain observation tool (CPOT). The biometric data obtained from the patient may be associated with physiological indicators of pain and may include one or more of a heart rate, a temperature, a blood pressure, a respiration rate, an oxygenation level, a motor activity level, a pupil constriction/dilation level, and sleep phase information. In some embodiments, the biometric data is obtained by the wearable device 122 (e.g., a smartwatch, a wristband, a fitness tracker, or a wearable medical device). Exemplary levels of some physiological indicators associated with pain are listed in Table 1. The physiological indicators associated with pain in Table 1 include systolic and diastolic blood pressure (BP), resting heart rate, oxygen saturation, and respiratory rate. For example, an elevated heart rate, an elevated blood pressure (either systolic and/or diastolic), a low oxygen saturation, and a low or elevated respiratory rate may be indications that the patient is experiencing pain at a particular level.

TABLE 1
Physiological Indicators of Pain

| | | | Resting | | |
|---|---|---|---|---|---|
| | Systolic | Diastolic | Heart | Oxygen | Respiratory |
| | BP | BP | Rate | Saturation | Rate |
| Normal | 100-120 | 60-80 | 60-100 | 95%-100% | 12-16 |
| Low | <90 | <60 | <60 | <90% | <12 |
| Elevated | 120-129 | <80 | >100 | | >25 |
| High Stage 1 | 130-139 | 80-89 | | | |
| Hypertension | | | | | |
| High Stage 2 | ≥140 | ≥90 | | | |
| Hypertension | | | | | |
| High Stage 3 | >180 | >120 | | | |
| Hypertension | | | | | |

At step 308, the dosage module 106 may match the pain level and/or inflammation level associated with the pain level indicators, and the user parameters with a dosage level indicated based on the dosage scheme generated at step 304. For example, the dosage module 106 determines based on the received pain level indicators that the patient has a particular pain level (e.g., level 8/10) on a standardized pain level scale. The determination of the pain level is done based on a pain level score received from the patient as well as biometric data obtained from the patient (e.g., one or more of the exemplary physiological indicators of pain illustrated in Table 1). The dosage module 106 then matches the particular pain level and the user parameters retrieved at step 302 with an appropriate dosage scheme generated at step 304. The appropriate dosage scheme includes an indication for a gradually decreasing amount of active ingredients over a period of time as well as an increasing, decreasing, or constant amount of active ingredients of other pain relief medications. In one embodiment, the dosage level and/or the dosage scheme is from the software kit database 104. The dosage scheme includes indications of doses for opioid compounds to be administered concurrently with cannabinoid compounds and/or other pain relief medications. For example, the dosage scheme includes indications of 25 mg of hydrocodone and 25 mg of CBD 4 times a day, and 500 mg of acetaminophen and 300 mg of ibuprofen twice a day to be administered to the patient on the day of the patient's surgery.

At step 310, based on the matching, the dosage module 106 may determine the dosage amount for the patient. For example, the dosage module 106 determines that the patient requires 25 mg of hydrocodone and 25 mg of CBD 4 times a day, and 500 mg of acetaminophen and 300 mg of ibuprofen twice a day, i.e., ((25×4)+(25×4)+(500×2)+(300×2)) mg=1800 mg of total pharmacologically relevant composition on the day of his surgery. In some embodiments, the dosage amount is determined based on, or extracted from, the software kit database 104.

In one embodiment, the dosage module 106 may determine which prescription is required for the patient. In one example embodiment, the prescription is dependent on the day associated with the surgery (such as the day of surgery or 2 days after the patient's surgery). Based on the determined dosage amount, the dosage module 106 may extract, at step 312, the indications of dosages from the software kit database 104. For example, the dosage module 106 extracts from the software kit database 104 that the patient requires 1800 mg of a pharmacologically relevant composition on the day of his surgery. Based on the extracted dosage indications from the software kit database 104, the dosage module 106 may send, at step 314, the dosage notification to the patient via the user device interface module 116. Alternatively, the dosage notification is sent to the third-party device 110 (e.g., to a physician or other caregiver). For example, the dosage module 106 sends a notification to the user device interface module 116 stating that the patient requires 1800 mg of a pharmacologically relevant composition on the day of his surgery, i.e., 25 mg of hydrocodone and 25 mg of CBD 4 times a day, and 500 mg of acetaminophen and 300 mg of ibuprofen twice a day. Thereafter, the program ends, at step 316.

Steps 306 through 314 can be repeated to update the determined dosage amount for the patient based on the received pain level indicators. For example, after opioid compounds and other pain relief medications were administered to the patient in accordance with the determined dosage amount at step 310, the patient's pain changes (e.g., increases or decreases). At step 306, the dosage module 106 receives updated pain level indicators from the client. At step 308, the dosage module 106 matches the updated pain level with an updated dose level, in accordance with the dosage scheme generated at step 306. The dosage module 106 further repeats the steps 310 through 316 accordingly.

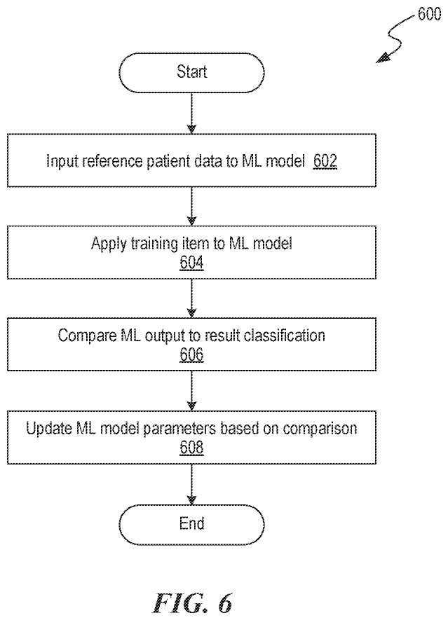

In some embodiments, the process performed by the dosage module 106, as described with reference to FIG. 3, further includes determining an addiction risk score for the patient based on the dosage scheme generated at step 304 and matching of the pain level with the dose level in accordance with the dosage scheme. As steps 306 and 308 are repeated (e.g., the patient's pain level and dose changes), the dosage module 106 re-determines the addiction risk score for the patient. The addiction risk score may be determined based on the patient data received at step 300 including gender, age, weight, height, medical conditions, and disposition. In some embodiments, the method also includes comparing the re-determined addiction risk score and the original addiction risk. For example, if the dose level is changed so that the re-determined addiction risk score increases beyond a pre-defined threshold, the dosage module 106 may re-determine the dosage level at step 310. In some embodiments, the addiction risk score is determined based on standardized drug screening and assessment tools. Such screening and assessment tools may include the Opioid Risk Tool-Opioid Use Disorder (ORT-OUD), Opioid Risk Tool (ORT), National Institute on Drug Abuse (NIDA) Drug Screening Tool, Tobacco, Alcohol, Prescription medication, and other Substances (TAPS) tool, Screening to Brief Intervention (S2BI), Drug Abuse Screening Test (DAST-10), or any other standardized drug screening and assessment tool. Determining addiction risk scores using machine learning are discussed in connection with FIG. 6.

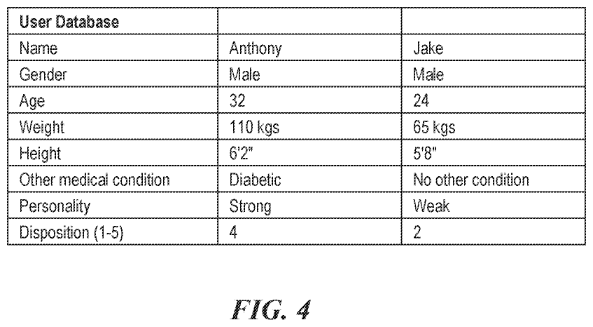

Functioning of the user device 112 will now be explained with reference to FIG. 4. The figure (i.e. FIG. 4) shows a screenshot of the user database 114 of the software kit 102 displayed on a computer device. The user database 114 may contain the data received by the user device interface module 116 from the user device 112 and/or the third-party device 110. The user database 114 contains the user admin data such as gender, age, weight, height, medical conditions, personality, and disposition. For example, FIG. 4 illustrates the user admin data for patients “Anthony” and “Jake.” The user admin data for patient Anthony includes gender—male, age—32, weight—110 kgs, height—6′2″, medical condition—diabetic, personality—strong, and disposition—4, and for patient Jake includes gender—male, age—24, weight—65 kgs, height—5′8″, medical condition—no other condition, personality—weak, and disposition—2. Additionally, the biometric data could include a heart rate, a respiration rate, oxygenation levels, blood-oxygen saturation (% SpO₂ or SaO₂) levels, motor activity levels, pupil size, sleep profile information, etc.

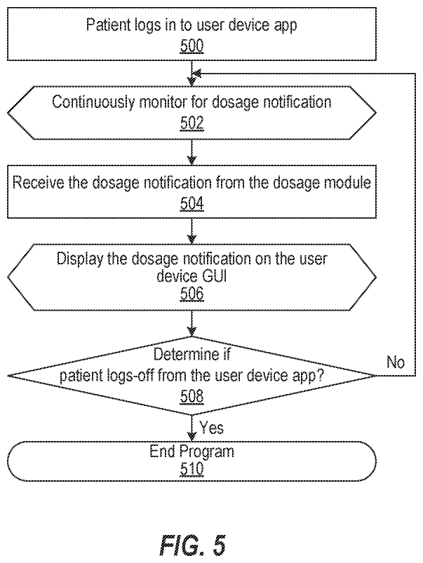

Functioning of the user device interface module 116 will now be explained with reference to FIG. 5. The figure (i.e. FIG. 5) is a flowchart illustrating processes performed by the user device interface module 116 of the user device 112. At step 500, the user device interface module 116 is triggered when the patient logs in either consciously or unconsciously (e.g., via biometric-activated switch) to the user device app 120 on the user device 112. The user device interface module 116 may enable the patient to extract dosages associated with a particular pain regimen of the patient. At step 502, after the patient logs in to the user device app 120, the user device interface module 116 may continuously monitor for the dosage notification from the dosage module 106. At step 504, the user device interface module 116 may receive the dosage notification from the dosage module 106. For example, the user device interface module 116 receives an indication from the dosage module 106 that the patient requires 1800 mg of a pharmacologically relevant composition on the day of his surgery, i.e., 25 mg of hydrocodone and 25 mg of cannabidiolic acid (CBDA) or cannabidiolic acid methyl ester 4 times a day, and 500 mg of acetaminophen and 300 mg of ibuprofen twice a day.

At step 506, the user device interface module 116 may display the dosage notification on the user device GUI 118. At step 508, the user device interface module 116 may determine if the patient logs off from the user device app 120. In one case, if the user device interface module 116 determines that the patient does not log off from the user device app 120, then the user device interface module 116 may return to step 502 to continuously monitor for the dosage notification from the dosage module 106. In another case, if the user device interface module 116 determines that the patient logs off from the user device app 120, then the program ends, at step 510.

In some embodiments, the user device interface module 116 may utilize the user device GUI 118 to enable the patient to input data. Further, the user device interface module 116 may store the input data in the user database 114 to allow the exchange of the input data with the dosage module 106. In one embodiment, the input data may include the user admin data such as, but not limited to, gender, age, weight, height, medical conditions, personality, and disposition. Further, the user device interface module 116 may prompt the patient with various questions to enable the patient to input data. For example, the user device interface module 116 may prompt the user with questions such as, but not limited to, “What is the user's gender?”, “What is the user's age?”, “What is the user's weight?”, “What is the user's height?”, “Does the user have any other medical condition?”, “What is the personality of the user?”, “What is the disposition level of the user?”, and “What is the pain intensity?”. In one embodiment, the user device interface module 116 may provide options in one or more questions from which the patient may choose such as, but not limited to, male and female options for the question “What is user's gender?”; very weak, weak, average, strong, and very strong options for the question “What is the personality of the user?”; a 1-5 rating meter for the question “What is the disposition level of the user?”; and a 1-10 rating meter for the question “What is the pain intensity?”.

In some embodiments of the present disclosure, the software kit 102 may include an indication of a pharmacologically relevant composition consisting of one or more cannabinoids, one or more non-opioid analgesics such as acetaminophen, and one or more nonsteroidal anti-inflammatory drugs (NSAIDs). The nonsteroidal anti-inflammatory drugs (NSAIDs) may be, for example, aspirin, ibuprofen, and naproxen. In some embodiments of the present disclosure, the software kit 102 may include an indication of a cannabinoid composition for pain management and opioid cessation. The cannabinoid composition may include one or more cannabinoids and a pain management composition, which may be pharmaceutical and/or botanical. In some embodiments of the present disclosure, the software kit 102 may include an indication of a pharmacologically relevant composition consisting of one or more cannabinoids, one or more anesthetic or sedative compounds such as amobarbital and ketamine, and/or one or more psychedelic drugs such as psilocybin (4-phosphoryloxy-N,N-dimethyltryptamine), psilocin, lysergic acid diethylamide (LSD), mescaline, and N,N-dimethyltryptamine (DMT). “A psychedelia compound” shall mean (1) any constituent extracted or derived from a plant, fungi or animal belonging to the genuses: Acacia, Alchornea, Amanita, Amsonia, Anadenanthera, Apocynum, Areca, Argyreia, Artemisia, Arundo, Aspidosperma, Banisteriopsis, Burkea, Calea, Calligonum, Calycanthus, Catha, Carex, Claviceps, Copelandia, Datura, Delosperma, Desfontainia, Desmanthus, Desmodium, Dictyoloma, Diplopterys, Dutaillyea, Echinopsis, Elaeagnus, Erigonum, Erythroxylum, Festuca, Guiera, Gymnacranthera, Hammada, Heimia, Horsfieldia, Ilex, Ipomoea, Iryanthera, Leonotis, Leptactinia, Lespedeza, Limonia, Lolium, Lophophora, Meconopsis, Melicope, Mimosa, Mitragyna, Mucuna, Nectandra, Newbouldia, Nicotiana, Nymphaea, Opuntia, Osteophloem, Panaeolus, Pandanus, Papaver, Passiflora, Pauridiantha, Peganum, Petalostylis, Phalaris, Phyllodium, Phyllomedusa, Picrasma, Pilocarpus, Plectocomiopsis, Prosopis, Psilocybe, Psychotria, Punica, Rhinella, Rivea, Salvia, Shepherdia, Simira, Strychnos, Tabernaemontana, Tabernanthe, Testulea, Tetradium, Trachelospermum, Tribulus, Uncaria, Urtica, Vepris, Vestia, Vinca, Virola, Voacanga, Zanthoxylum, and Zygophyllum; (2) any compounds in the following chemical classes: arylcyclohexylamines, beta-carbolines, cathinones, ergolines, indole alkaloids, lysergamides, methylxanthine alkaloids, muscimol (and precursors), phenethylamines, salvinorins, tryptamines, Phyllomedusa peptides, and generally any compound or class of compounds categorized as “hallucinogenic substance” in schedules 1-5 of the United States Controlled Substance Act or analogues thereof; or (3) any compounds or formulations that exhibit central nervous system (CNS) activity at adenosinergic, adrenergic, cannabinergic, dopaminergic, GABA, NMDA, norepinephrine, and serotoninergic (e.g. 5-HT_{2A} and 5-HT_{1A}) receptors.

In some embodiments of the present disclosure, the software kit 102 for opioid cessation includes indications of compositions of opioids and cannabinoids in various proportions. The proportion of opioids may gradually decrease throughout the software kit 102 compositions as the proportions of cannabinoids gradually increase. The cannabinoids may be selected for pain management therapy. The final composition of the software kit 102 may contain zero or a negligible quantity of opioids.

In some embodiments of the present disclosure, the software kit 102 is configured to match cannabinoids and other cannabis components with nutritional supplements for synergistic and/or additive effects. The matching may include assessing the intended effect of a nutritional supplement and matching it with the cannabinoid extract with the most synergistic properties.

In some embodiments of the present disclosure, the software kit 102 may be programmed either by a human or by an artificial intelligence mechanism to drastically change the course of the dosage regimen in response to a deleterious signal or pattern from the data interface. For example, low SpO2 levels may lock dispensing functions of the entire pharmacologically active composition or one or more of the component elements to avoid serious adverse events.

In some embodiments of the present disclosure, the software kit 102 may include an indication for a multi-compartment capsule with a cannabinoid extract and a nonsteroidal anti-inflammatory drug. The patient may take both compartments of the capsule simultaneously or split the capsule to individually select only cannabinoid or only NSAID dosages.

The functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments. Included examples of classes of pharmacologically active compounds and compositions are intended for illustrative purposes and are not intended to be limited by those explicitly disclosed examples.

### Processes for Training Machine Learning Models

FIG. 6 illustrates a flowchart showing a method 600 for training a machine learning model, in accordance with some embodiments. Machine learning models, such as neural networks, can be trained to produce types of results. A neural network can be trained by obtaining, at block 602, a quantity of “training items,” where each training item includes input similar to input the model will receive when in use and a corresponding scored result. At block 604, the input from each training item can be supplied to the model to produce a result. At block 606, the result can be compared to the scored result. At block 608, model parameters can then be updated, based on how similar the model result is to the scored result and/or whether the score is positive or negative. The method 600 can be used to generate one or more trained machine learning models for outputting any of the following: dosing schemes for a combination of opioid compound(s) with cannabinoid compound(s), and/or other therapeutic compound(s), therapeutically effective amounts corresponding to a particular pain level (e.g., on a standard pain scale) over a period of time, prediction of a future pain level within a period of time, therapeutic effect, risk scores, etc. The steps of the method 600 can be selected based on the inputs and desired output and are discussed below.

At block 602, model input can include, without limitation, reference patient data and reference drug scheme data. The reference patient data (e.g., data from the software kit database 104 of FIG. 1) may include pain level, gender, age, weight, height, medical conditions, personality, disposition, and so forth. The reference patient data may further include data associated with pain alleviation, opioid addiction, cannabinoid addiction, opioid efficacy in alleviating pain, and/or cannabinoid efficacy in alleviating pain. The reference drug scheme data may include indications of opioid compound dosages administered to a patient in accordance with an opioid tapering dosage scheme and indications of cannabinoid and/or other pain relief compound dosages administered to the patient in accordance with a respective cannabinoid and/or other pain relief compound dosage scheme. The training data input can be classified and/or paired with results to create training items. The classification can be selected based on the model characteristics and output. The results for training items can be, for example, patient feedback to model outputs (e.g., a pain score on a standardized scale), healthcare-provided suggestion feedback (e.g., whether the healthcare-accepted model provided recommendations completely, or made certain changes, or disregarded), biometric data associated with physiological indicators of pain (e.g., a heart rate, a temperature, a blood pressure, a respiration rate, an oxygenation level, a motor activity level, a pupil constriction/dilation level, and sleep phase information) analyzed to determine a pain score, the existence of certain positive or negative patient experiences with respect to pain alleviation, patient-reported measures and functional scores using standard scoring systems such as SF-36, OSI, NDI, WOMAC, EQ-5D, PROMIS, Oxford Hip and Knee scores, or similar outcome scoring systems, can be utilized as a proxy measure for pain and function, and can be used to refine the personal dosing schemes, settings, or the like. In addition, patient-reported measures and functional scores using standard scoring systems such as SF-36, OSI, NDI, WOMAC, EQ-5D, PROMIS, Oxford Hip and Knee scores, or similar outcome scoring systems, can be utilized as a proxy measure for pain and function, and can be used to refine the personal dosing schemes, settings, etc. The biometric data associated with physiological indicators may include objective data, such as personal data collected by biometric or wearable devices. The user feedback may result in refined personal dosing schemes, recommended products, recommended settings, etc. The data discussed in connection with FIG. 3 can be used as model input, model selection, etc. The input, subject scoring, and other information can be collected via, for example, a user device (e.g., user device 112 of FIG. 1), input/output devices (e.g., input/output devices 725 of FIG. 7, etc.). In some embodiments, user feedback is aggregated. Aggregated user feedback may be used to develop and refine a heuristic algorithm to provide schemes, recommendations, etc., to new patients, patients using new substances, or the like.

At block 604, input from each training item can be supplied to the model to produce a result or output. The output can be converted to arrays of integers that, when provided to the machine learning model, produce values that specify dosing schemes for opioid compounds to be administered concurrently with cannabinoid and/or other pain relief medication compounds. Any number of models can be generated to recommend dosing schemes.

At block 606, results can be compared to the scored result or result classification. For example, result dosing schemes can be compared to actual dosing schemes used by reference patients that produced the training item. The model can correlate the dosage schemes information to predicted patient experience. The dosage schemes can indicate any combinations of opioids, cannabinoids, and other pain relief medication compounds administered with any active ingredient amounts (e.g., by concentrations, volumes, weights), any types of compound formulations (e.g., pills, capsules, tablets, transdermal patches, injections, tinctures, smokable herbs, vaporizers, or any other form), dosage frequency, time period of administering the compounds, etc.

At block 608, model parameters can be updated, based on how similar the model result is to the scored result and/or whether the score is positive or negative. The model parameters can then be adjusted so that the model output is more like the prior dosing scheme and patient experience if that prior patient experience was a success, or less like the prior dosing scheme if the prior patient experience was unsuccessful (e.g., pain alleviation not successful, other undesired side effects, etc.). The amount of adjustment to the model parameters can be a function of how different the model prediction was from the actual dosing scheme used and/or the level of success or failure of the product usage. Machine learning models can be trained to produce various results, such as to provide fast reduction of the opioid compound in the dosage scheme, target alleviation of pain, target types of compound formulations, target combination of the opioid compensating compounds including cannabinoid and/or other pain-relieving compounds, or the like. Models can be grouped or classified based on patient characteristics, such as patient sensitivity.

The method 600 can generate dispensing schemes based on a patient's profile, previous sensor data for that patient, and/or information and/or sensor data from a plurality of other patients. In some embodiments, a health state is quantified as a score or metric representing the patient's overall health status and/or risk, which can be generated based on any suitable combination of sensor data and/or other data. In some embodiments, patient-specific settings or recommendations may be based upon, for example, the health state, specific patient's experience feedback, etc. The patient experience feedback may query the patient for a variety of parameters (e.g., the standardized pain scale scores, query to overall well-being of the patient, etc.). Questions may solicit information regarding, for example, the patient's experience with respect to pain alleviation, the patient's mood, the patient's fatigue level/sleep deprivation, the patient's appetite, the patient's activity and mobility, etc. For example, a model can be trained using sets of patient feedback and corresponding scores for usage. The sets of patient feedback and corresponding scores can be in accordance with standard scoring systems such as SF-36, OSI, NDI, WOMAC, EQ-5D, PROMIS, Oxford Hip and Knee scores, or similar outcome scoring systems.

In some embodiments, multiple machine learning training procedures can be performed. Example procedures can include obtaining suitable training data sets associated with a result, applying each training data set to the model, and updating model parameters based on comparison of the model result to the training set result. Each model can be designed for a different result. A neural network can be trained by obtaining a quantity of “training items or data sets,” where each training item or data set includes input similar to input the model will receive when in use and a corresponding scored result. The input from each training item/data set can be supplied to the model to produce a result. The result can be compared to the scored result. Model parameters can then be updated based on how similar the model result is to the scored result and/or whether the score is positive or negative. A training procedure can include clustering, predictive analysis, etc., as discussed above. The training procedure can be selected based on the amount, quality, and/or characteristics of the data.

The method 600 can generate risk scores for the patient. In some embodiments, the method 600 includes determining an addiction risk score for the patient using a machine learning model. The machine learning model can be trained using training data sets containing patient risk factor data (e.g., history of drug abuse, methadone use, chronic opioid use, comorbid illness, number of physicians, etc.), addiction outcomes (e.g., whether a patient become addicted), and corresponding scores (e.g., addiction risk scores). In some training routines, the training data sets include, without limitation, addiction outcomes, standardized drug screening and assessment scores. Such screening and assessment scores may include, scores from one or more of the ORT-OUD, ORT, NIDA Drug Screening tool, TAPS tool, S2BI test, DAST-10, or any other standardized drug screening and assessment tool. The trained machine learning model can analyze patient data (e.g., patient data received at step 300 of FIG. 3) to identify risk scores, predicted addition outcomes, and other addition related predictions. A dosage scheme can be generated or adjusted based on the based on the addiction risk score for the patient. This allows a healthcare professional monitor addition risk throughout treatment. If the patient becomes addicted, the patient data set can be used to retrain the machine learning model.

The machine learning model can also be trained to generate an adjusted dosage scheme based on the addiction risk score to, for example, keep an addition risk score below an threshold level. The threshold level can be set a physician, healthcare provider, or user can be related to usage time. For example, a threshold level for 1 month of continued opioid use can be equal to or less than 2.5%, 5%, 10% likelihood of continued opioid use. A threshold level for 1 year of continued opioid use can be equal to or less than 5%, 10%, 20%, or 30% addiction risk score for likelihood of continued opioid use. The planned usage information can be inputted into the model trained machine learning model such that the trained machine learning model generates a dosage scheme with an addiction risk score at or below the threshold level.

### Computing Systems and Devices

In an illustrative embodiment, any of the operations, processes, etc., described herein can be implemented as computer-readable instructions stored on a computer-readable medium. The computer-readable instructions can be executed by a processor of a mobile unit, a network element, and/or any other computing device.

Some implementations can be operational with numerous other computing system environments or configurations. Examples of computing systems, environments, and/or configurations that may be suitable for use with the technology include, but are not limited to, personal computers, server computers, handheld or laptop devices, cellular telephones, wearable electronics, gaming consoles, tablet devices, multiprocessor systems, microprocessor-based systems, set-top boxes, programmable consumer electronics, network PCs, minicomputers, mainframe computers, distributed computing environments that include any of the above systems or devices, or the like.

FIG. 7 is a block diagram of a computing system 700 (also referred to as “a system”) that may be used to implement features of some embodiments of the disclosed technology. In some embodiments, the computing system corresponds to the user device 112, the third-party device 110, and/or a computer device running the software kit 102. The computing system 700 may be used to implement any of the entities, components, or services depicted in the examples of FIGS. 1-6 (and any other components described in this specification).

The computing system 700 may include one or more central processing units (“processors”) 705, memory 710, input/output devices 725 (e.g., keyboard and pointing devices, buttons, display devices, etc.), storage devices 720 (e.g., disk drives), and network adapters 730 (e.g., network interfaces) that are connected to an interconnect 715. The interconnect 715 is illustrated as an abstraction that represents any one or more separate physical buses, point-to-point connections, or both connected by appropriate bridges, adapters, or controllers. The interconnect 715, therefore, may include, for example, a system bus, a Peripheral Component Interconnect (PCI) bus or PCI-Express bus, a HyperTransport or industry standard architecture (ISA) bus, a small computer system interface (SCSI) bus, a universal serial bus (USB), an IIC (I2B) bus, or an Institute of Electrical and Electronics Engineers (IEEE) standard 1394 bus.

The memory 710 and storage devices 720 are computer-readable storage media that may store instructions that implement at least portions of the described technology. In addition, the data structures and message structures may be stored or transmitted via a data transmission medium, such as a signal on a communications link. Various communications links may be used, such as the internet, a local area network, a wide area network, or a point-to-point dial-up connection (e.g., a Bluetooth connection). Thus, computer-readable media can include computer-readable storage media (e.g., “non-transitory” media) and computer-readable transmission media.

The instructions stored in memory 710 can be implemented as software and/or firmware to program the processor(s) 705 to carry out the actions described above. In some embodiments, such software or firmware may be initially provided to the computing system 700 by downloading it from a remote system through the computing system 700 (e.g., via network adapter 730).

The technology introduced herein can be implemented by, for example, programmable circuitry (e.g., one or more microprocessors) programmed with software and/or firmware, or entirely in special-purpose hardwired (non-programmable) circuitry, or in a combination of such forms. Special-purpose hardwired circuitry may be in the form of, for example, one or more ASICs, PLDs, FPGAs, etc.

The input/output devices 725 may include input devices such as keyboards, mice, trackpads, trackballs, touchpads, touch mice, multi-touch touchpads and touch mice, microphones, multi-array microphones, drawing tablets, cameras, single-lens reflex (SLR) cameras, digital SLR (DSLR) cameras, CMOS sensors, accelerometers, infrared optical sensors, pressure sensors, magnetometer sensors, angular rate sensors, depth sensors, proximity sensors, ambient light sensors, gyroscopic sensors, or other sensors. Output devices may include video displays, graphical displays, speakers, headphones, inkjet printers, laser printers, and 3D printers. Devices may include a combination of multiple input or output devices, including, for example, touch screens, physical buttons, microphones, fingerprint readers, accelerometers, vibration devices, etc. Some devices allow gesture recognition inputs by combining some of the inputs and outputs. Some devices allow for facial recognition, which may be utilized as an input for different purposes, including authentication and other commands. Such devices allow for voice recognition and inputs, including, for example, Microsoft Kinect, Siri for iPhone by Apple, Google Now, or Google Voice Search. Additional mobile devices have both input and output capabilities, including, for example, haptic feedback devices, touchscreen displays, or multi-touch displays. Touchscreen, multi-touch displays, touchpads, touch mice, or other touch sensing devices may use different technologies to sense touch, including, for example, capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices may allow two or more contact points with the surface, allowing advanced functionality including, for example, pinch, spread, rotate, scroll, or other gestures. Some touchscreen devices, including, for example, Microsoft PixelSense or Multi-Touch Collaboration Wall, may have larger surfaces, such as on a tabletop or a wall, and may also interact with other electronic devices. Some I/O devices, display devices, or groups of devices may be augmented reality devices.

FIG. 8 is a block diagram illustrating an overview of a system 800 on which some implementations of the disclosed technology can operate. In some embodiments, the system 800 corresponds to the user device 112, the third-party device 110, and/or a computer device running the software kit 102. The system 800 may be used to implement any of the entities, components, or services depicted in the examples of FIGS. 1-6 (and any other components described in this specification).

The system 800 can include one or more sensors 818 and input devices 820 that provide input to a processor(s) 810 (e.g., CPU(s), GPU(s), HPU(s), etc.), notifying it of, for example, adverse event(s), operation, and/or actions. The input can be mediated by a hardware controller that interprets the signals received from the input device and communicates the information to the processors 810 using a communication protocol. The processors 810 can be a single processing unit or multiple processing units in a device or distributed across multiple devices. The processors 810 can be coupled to other hardware devices, for example, with the use of a bus, such as a PCI bus or SCSI bus. The processors 810 can communicate with a hardware controller for devices, such as for a display 830. Display 830 can be used to display text, graphics, indicators, etc. In some implementations, display 830 provides graphical and/or textual visual feedback (e.g., dosage information, pain level scores, biometric data, other user-associated data) to a patient. In some implementations, display 830 includes the input device as part of the display, such as when the input device is a touchscreen. Examples of display devices are an LCD display screen, an LED display screen, a projected or augmented reality display, such as a heads-up display device or a head-mounted device, and so on. For example, an augmented reality display can display dosing information in a virtual environment (e.g., a virtual environment for meditation, therapy, etc.). Other I/O devices 840 can also be coupled to the processor, such as a user device (e.g., user device 112), network card, video card, audio card, USB, firewire or other external device, camera, speakers, etc. In some embodiments, the system 100 also includes a communication device 844 capable of communicating wirelessly or wire-based with a network node. The communication device can communicate with another device or a server through a network using, for example, TCP/IP protocols. The system 800 can utilize the communication device to distribute operations across multiple network devices.

The processors 810 can have access to a memory 850 in a device or distributed across multiple devices. A memory includes one or more of various hardware devices for volatile and non-volatile storage, and can include both read-only and writable memory. For example, a memory can include random access memory (RAM), various caches, CPU registers, read-only memory (ROM), and writable non-volatile memory, such as flash memory, hard drives, floppy disks, CDs, DVDs, magnetic storage devices, tape drives, and so forth. A memory is not a propagating signal divorced from underlying hardware; a memory is thus non-transitory. Memory 850 can include program memory 860 that stores programs and software, such as an operating system 862, a dispensing or dosing system 864 (“dispensing system 864”), and other application programs 866. Memory 850 can also include data memory 870, e.g., authentication information (e.g., cartridge authentication, user authentication, liquid composition authentication, etc.), biometric data, compound data, cartridge data, notification data, user personal health information, configuration data, settings, user options or preferences, etc., which can be provided to the program memory 850 or any element of the system 800.

The systems (e.g., systems 700 and 800) can be part of the system 100 of FIG. 1 and other systems or components disclosed herein. For example, the user device 112, the third-party device 110, and/or the wearable device 122 can include all or a portion of the systems 700 (FIG. 7) or 800 (FIG. 8). As another example, the software kit 102 may be a software program or an application running on the systems 700 (FIG. 7) or 800 (FIG. 8). For example, the memory 850 of the system 800 (FIG. 8) may include instructions that, when executed by the processors 810, cause the system 800 to perform any of the processes described with respect to the software kit 102, the user device 112, the third-party device 110, and/or the wearable device 122, as described above with respect to FIGS. 1-6.

The systems and devices disclosed herein can be configured for machine learning model(s), such as example machine learning models discussed in connection with FIG. 6. The machine learning models can be of various types, such as Convolutional Neural Networks (CNNs), other types of neural networks (e.g., fully connected), decision trees, forests of classification trees, Support Vector Machines, etc. Machine learning models can be trained to produce particular types of results, operations, etc. For example, a training procedure can include obtaining suitable training items with input associated with a result, applying each training item to the model, and updating model parameters based on comparison of the model result to the training item result. The machine learning model(s) can be generated by, for example, the cloud 108 of FIG. 1 using data from the software kit database 104 of FIG. 1.

### Exemplary Embodiments of the Present Disclosure

In accordance with some embodiments, a method for reducing opioids in a pain-modulating regimen of a patient is performed by a computer system (system 700 in FIG. 7 or system 800 in FIG. 8). The method includes generating, by a computer system, a dosage scheme (e.g., the dosage scheme illustrated in FIG. 2) for the patient based on parameters associated with a health condition of the patient (e.g., patient data illustrated in FIG. 4). The dosage scheme includes a first dosage scheme for an opioid compound and a first dosage scheme for a cannabinoid compound. The opioid and the cannabinoid are to be administered to the patient concurrently over a period of time (e.g., a week, two weeks, a month). The first dosage scheme for the opioid compound includes a first plurality of dosages with a decreasing amount of an active ingredient in the opioid compound over the period of time. The method also includes determining an addiction risk score for the patient based on the dosage scheme and determining whether to adjust the dosage scheme based on the addiction risk score for the patient. In response to determining to adjust the dosage scheme, the method includes generating an adjusted dosage scheme based on the pain level indicators.

In some embodiments, the method also includes receiving pain level indicators associated with the patient. The pain level indicators include a first pain level indicator based on an input from the patient and a second pain level indicator based on biometric data obtained by a wearable device (e.g., wearable device 122 in FIG. 1). The wearable device is configured to be worn by the patient. The method includes determining, by the computer system, whether to adjust the dosage scheme based on the pain level indicators. In response to determining to adjust the dosage scheme, the method includes generating an adjusted dosage scheme based on the pain level indicators. The adjusted dosage scheme includes a second dosage scheme for the opioid compound. The adjusted dosage scheme is different from the first dosage scheme for the opioid compound to be administered to the patient.

In some embodiments, the method includes determining based on the received pain level indicators at least one relationship between pain-reduction efficacy of the cannabinoid compound and pain-reduction efficacy of the opioid compound. The adjusted dosage scheme is generated based on the determined at least one relationship and has a predicted pain score that is less than a maximum pain score. For example, based on the input received from the patient and the biometric data obtained by the wearable device, and the dosage of the cannabinoid compound or the opioid compound, the system determines the pain-reduction efficacy of the cannabinoid or opioid compound, respectively. The determined relationship may then be used to predict a pain score for a patient receiving cannabinoid and/or opioid compounds according to the dosage scheme or the adjusted dosage scheme.

In some embodiments, the first dosage scheme for the cannabinoid compound includes a second plurality of dosages with an increasing amount of an active ingredient in the cannabinoid compound over the period of time. The cannabinoid compound dosages are thereby configured to compensate for the reducing amount of the active ingredient in the cannabinoid compound over the period of time.

In some embodiments, the dosage scheme further includes a third dosage scheme for a pain relief medication to be administered to the patient concurrently. The pain relief medication can include non-steroidal anti-inflammatory drugs (e.g., aspirin, ibuprofen, and naproxen) and other drugs, plant-derived extracts and complex mixtures (e.g., kratom, turmeric, white willow, and Boswellia), and mind-body techniques (e.g., acupuncture and meditation).

In some embodiments, the biometric data obtained by the wearable device includes one or more of a heart rate, a temperature, a blood pressure, a respiration rate, an oxygenation level, a motor activity level, a pupil constriction/dilation level, and sleep phase information. The biometric data may correspond to physiological indicators associated with pain, such as those included in Table 1.

In some embodiments, the method includes transmitting, by the computer system to a user device (e.g., the user device 112 in FIG. 1) associated with the patient, instructions to administer the opioid compound and the cannabinoid compound to the patient in accordance with the adjusted dosage scheme. The transmitting is performed in response to receiving the pain level indicators associated with the patient. In some embodiments, the instructions include times, amounts, types, and methods of administering the opioid compound and the cannabinoid compound to the patient.

In some embodiments, the opioid compound and the cannabinoid compound are independently administered as pills, capsules, tablets, transdermal patches, injections, tinctures, smokable herbs, or as an inhalable vapor. For example, the opioid compound is administered as an injection and the cannabinoid is administered as an inhalable vapor. As another example, both the opioid and the cannabinoid are administered as tablets.

In some embodiments, the dosage scheme and the adjusted dosage scheme are configured to gradually reduce the opioid compound from the pain-modulating regimen of the patient by replacing the opioids with the cannabinoid compound. For example, as shown in FIG. 2, the opioid dose is reduced gradually from the day of the surgery to 8 days post-surgery.

In some embodiments, the method further includes determining, by the computer system, whether an amount of opioid administered to the patient in accordance with the dosage scheme or the adjusted dosage scheme is above a threshold limit. For example, the threshold limit can correspond to an amount of opioid compound that is defined by, for example, a health official, or by a health care provider (e.g., a hospital, a physician, an insurance company, or government agencies (e.g., Centers for Disease Control and Prevention (CDC), Federal Drug Administration (FDA), etc.) to be, for example, a maximum amount of opioid that a user can consume. The threshold limit may be defined per dosage unit, per day (e.g, defined daily dose), per week, etc. The threshold limit may depend on the patient's biometric information such as weight, age, health conditions, or the addiction risk score. The method further includes reducing the amount of opioid compounds administered to the patient in an instance where the amount of opioids is above the threshold limit.

In some embodiments, the computer system generates the adjusted dosage scheme partially based on an input received from a user of the computer system. For example, a physician may provide an input to adjust (e.g., increase or decrease) the amount of opioids to be administered to the patient, dosage times, or dosage intervals. In some instances, the computer system determines whether the amount of opioids administered to the patient is above the threshold limit described above. In accordance with a determination that the amount of opioid administered to the patient is above the threshold limit, the computer system provides an indication (e.g., a warning icon or a pop-up window) to the user. Such indication is configured to prevent instances of overprescribing.

In some embodiments, the computer system further provides indications (e.g., icons, pop-up windows, or other indications) indicating when the dosage scheme or the adjusted dosage scheme does not correspond to an appropriate medical billing code. For example, administering opioids under a medical billing code for surgery may be appropriate while administering opioids under a medical billing code for a toothache is not appropriate. The medical billing codes may further include limitations for administering opioids. For example, a medical billing code for surgery may allow a certain amount of opioids to be administered for a certain period of time to be covered by federal or state health coverages or by health insurance companies. The computer system will provide an indication in an instance where the amount of opioids is higher than the covered amount. Such indication is configured to prevent the administration of medications that are not covered by federal or state health coverages or by health insurance companies. In some embodiments, the computer system can generate dosing schemes based on reimbursement information, such as reimbursement billing and coding. For example, the computer system can determine dosage schemes ensure compliance and payment coverage by Centers for Medicare & Medicaid Services (CMS), insurance provider, or the like. The computer system can search for and retrieve, for example, codes, dosing limits, threshold limits, and other information from third party databases, such as FDA databases, CMS databases, CDC databases, etc. Patient dosing scheme can also be generated based on reporting requirements.

In some embodiments, the method further includes determining an addiction risk score for the patient based on the dosage scheme and determining an adjusted addiction risk score for the patient based on the adjusted dosage scheme (e.g., as described with respect to FIG. 3). The addiction risk score may be determined based on the patient data including pain intensity, gender, age, weight, height, medical conditions, personality, and disposition (e.g., history of prior addictions). The method also includes comparing the addiction risk score and the adjusted addiction risk score to determine whether the second dosage scheme meets at least one addiction risk criterion. In some embodiments, the addiction risk score is determined based on standardized drug screening and assessment tools. Such screening and assessment tools may include the Opioid Risk Tool-Opioid Use Disorder (ORT-OUD), Opioid Risk Tool (ORT), National Institute on Drug Abuse (NIDA) Drug Screening Tool, Tobacco, Alcohol, Prescription medication, and other Substances (TAPS) tool, Screening to Brief Intervention (S2BI), Drug Abuse Screening Test (DAST-10), or any other standardized drug screening and assessment tool.

An addiction risk criterion could include a threshold for reduction of the addiction risk score. For example, meeting the addiction risk criterion includes the requirement that the addiction risk score is maintained at or above the addiction risk score “medium” or below a 30% likelihood of developing an addiction. As another example, the addiction risk criterion is met when the addiction risk score does not increase by more than 20% (e.g., the addiction risk criterion is met when the addiction risk assessment score is increased by 20% or less). In some embodiments, the addiction risk criterion includes a time frame. For example, the addiction risk criterion is met when the patient has a likelihood of developing an addiction below the threshold over a period of time (e.g., the addiction risk criterion is expected to be maintained below the threshold for a week or a month).

In accordance with some embodiments, a system (e.g., the system 100 in FIG. 1) for eliminating opioids from a pain-modulating regimen of a patient is disclosed. The system includes a computer device programmed to be in communication with a user device and a wearable device associated with the patient. The system is configured to perform the disclosed methods.

In some embodiments, the user device (e.g., the user device 112 in FIG. 1) is configured to receive instructions to administer the opioid compound and the cannabinoid compound to the patient in accordance with the dosage scheme and the adjusted dosage scheme and display the instructions to the patient.

In some embodiments, the wearable device (e.g., the wearable device 122) is configured to obtain the biometric data from the patient and transmit the biometric data to the computer device, thereby enabling the computer device to generate the adjusted dosage scheme. In some embodiments, obtaining the biometric data includes detecting, by one or more sensors of the wearable device, one or more of a heart rate, a temperature, a blood pressure, a respiration rate, an oxygenation level, a motor activity level, a pupil constriction/dilation level, and sleep phase information of the patient. The wearable device may be a smartwatch, a wristband, a fitness tracker, or a wearable medical device.

In accordance with some embodiments, a method for administering drugs to a patient includes generating, by a computer system, an initial dosage scheme (e.g., step 304 in FIG. 3) for the patient based on parameters associated with a health condition of the patient. The initial dosage scheme includes a first dosage scheme for a first drug and a first dosage scheme for a second drug to be administered to the patient concurrently over a period of time. The first dosage scheme for the first drug includes a first plurality of dosages with a decreasing amount of an active ingredient in the first drug over the period of time. The method includes receiving, by the computer system, one or more health condition indicators associated with the patient. The one or more health condition indicators include an indicator based on an input from the patient. The method also includes generating, by the computer system, a subsequent dosage scheme based on the one or more health condition indicators.

In some embodiments, the method further includes administering a first set of dosages of the opioid compound and a first set of dosages of the cannabinoid compound to the patient in accordance with the dosage scheme. The method also includes administering a second set of dosages of the opioid compound and a second set of dosages of the cannabinoid compound to the patient in accordance with the adjusted dosage scheme. Administering can be done by a device coupled or in communication with the computer system. For example, the system 100 includes or is in communication with a drug administration system or device. The drug administration system or device can be configured to control the quantity, volume, concentration, etc., of drugs administered to the patient. Alternatively, the administering is done by a person (e.g., the patient or the patient's caretaker).

In accordance with some embodiments, a method for opioid management in a pain-modulating regimen for a patient includes receiving, by a computing system, patient data of a patient. The method includes generating, by at least one trained cannabinoid dosing machine learning model, a pain relief regimen based on the received patient data. For example, the pain relief regimen is generated by a trained cannabinoid dosing machine learning model described with respect to FIG. 6. The pain relief regimen includes an opioid tapering dosage scheme and a cannabinoid dosage scheme (e.g., as shown in FIG. 2). The cannabinoid is designed to compensate for opioid tapering of the opioid tapering dosage scheme such that the pain relief regimen effectively alleviates pain of the patient.

In some embodiments, the pain relief regimen includes a first dosage scheme including a first opioid tapering dosage scheme for an opioid compound and a first cannabinoid dosage scheme for a cannabinoid compound to be administered to the patient concurrently over a period of time.

In some embodiments, the method further includes receiving, by the computing system, one or more pain level indicators for the patient. The pain level indicators may include patient feedback or biometric data including physiological indicators of pain (e.g., Table 1). The method includes determining whether to adjust the pain relief regimen based on the received one or more pain level indicators. In response to determining to adjust the pain relief regimen, the method includes generating an adjusted effective pain relief regimen based on a relationship between the one or more pain level indicators and an addiction risk score. An addiction risk score refers to a likelihood of a patient developing an addiction to, for example, opioids. The addiction score can include a verbal evaluation. For example, the addiction risk can be assessed as “high,” “medium,” or “low.” Alternatively, the addiction risk can be assessed as a numerical value. For example, 10%, 20%, 30%, 40%, 50%, 60%, 70%, 80%, or 90% likelihood of developing an addiction. The addiction risk can be determined based on the patient's disposition to addiction including, for example, a history of addictive behavior, genetic disposition to addiction or drug or alcohol abuse, electroencephalogram (EEG) patterns, or functional magnetic resonance imaging (fMRI) patterns, etc. The addiction risk can also be determined based on medical conditions. For example, medical conditions associated with chronic pain or extremely high pain may increase the likelihood of developing an addiction. The adjusted dosage scheme includes a modified opioid tapering dosage scheme for reducing a risk of opioid addiction by the patient.

In some embodiments, retraining the at least one trained cannabinoid dosing machine learning model is performed by using a plurality of reference patient sets that include pain data and opioid misuse data.

In some embodiments, the method further includes receiving pain data of the patient associated with the pain relief regimen. The pain data can be received as an input from the patient or as biometric data (e.g., measured by the wearable device 122). The method also includes using the at least one trained cannabinoid dosing machine learning model to analyze the received pain data and to identify, based on the analysis of the received pain data, one or more patient-specific correlations between opioid dosing of the opioid tapering dosage scheme and cannabinoid dosing of the cannabinoid dosing scheme. The method includes generating an adjusted pain relief regimen based on the identified one or more patient-specific correlations to alleviate pain of the patient.

In some embodiments, generating the pain relief regimen includes predicting pain levels of the patient over a period of time and determining, based on the predicted pain levels, the opioid tapering dosage scheme for at least one opioid compound and the cannabinoid dosage scheme for at least one cannabinoid compound.

In some embodiments, the method also includes determining a reduction of pain alleviation associated with opioid tapering of the opioid tapering dosage scheme over a period of time. The method includes determining the cannabinoid dosage scheme for providing pain alleviation that is substantially proportional to the reduction of pain alleviation associated with opioid tapering of the opioid tapering dosage scheme over the period of time.

In some embodiments, the method further includes comparing the patient data to a plurality of reference patient data sets to identify one or more similar patient data sets in the plurality of reference patient data sets. The similarity is based on at least one of pain alleviation, opioid addiction, cannabinoid addiction, opioid efficacy, or cannabinoid efficacy. The method includes selecting a subset of the one or more similar patient data sets that includes data indicative of a favorable pain relief and non-addiction outcome. The method also includes identifying, for at least one similar patient data set of the selected subset, one or more dosing parameters associated with the favorable pain relief and non-addiction outcome. The method includes using the one or more dosing parameters to generate the pain relief regimen.

### Example 1. CBD-Facilitated Taper

This example demonstrates that CBD is used to facilitate taper of opioid (oxycodone) use in opioid-naive patients, mildly opioid-dependent patients, and moderately opioid-dependent patients. For dosing schedules, the following abbreviations are used: BID=2 times per day, every 12 hours; PO=by mouth; QD=daily; Q4H=every 4 hours; Q6H=every 6 hours; TID=3 times per day, every 8 hours.

Tables 2-4 are examples of dosing schedules for illustration purposes only. Depending on the conditions and responses of individual patients, the dosing schedules can be adjusted accordingly.

TABLE 2
CBD-Only Facilitated Taper for Opioid-Naive Patient

| | Oral Dosing Medication Taper Sample Schedule | | | | | |
|---|---|---|---|---|---|---|
| | Week 1 | Week 2 | Week 3 | Week 4 | Week 5 | Week 6 |
| Acetaminophen | 3000 mg QD | 3000 mg QD | 3000 mg QD | 2000 mg QD | 2000 mg QD | 2000 mg QD |
| | divided TID | divided TID | divided TID | divided TID | divided TID | divided TID |
| Ibuprofen | 600 mg PO | 600 mg PO | 400 mg PO | 400 mg PO | 200 mg PO | 200 mg PO |
| | TID | TID | TID | TID | TID | TID |
| Oxycodone | 5 mg Q4 H | 2.5 mg Q4 H | 0 mg | 0 mg | 0 mg | 0 mg |
| | (20 mg QD) | (10 mg QD) | | | | |
| Cannabinol | — | 300 mg QD | 300 mg QD | 300 mg QD | 300 mg QD | 300 mg QD |
| (CBD) | | divided TID | divided TID | divided TID | divided TID | divided TID |

TABLE 3
CBD-Only Facilitated Taper for Mildly Opioid-Dependent Patient

| | Oral Dosing Medication Taper Sample Schedule | | | | | |
|---|---|---|---|---|---|---|
| | Week 1 | Week 2 | Week 3 | Week 4 | Week 5 | Week 6 |
| Acetaminophen | 3000 mg QD | 3000 mg QD | 3000 mg QD | 2000 mg QD | 2000 mg QD | 2000 mg QD |
| | divided TID | divided TID | divided TID | divided TID | divided TID | divided TID |
| Ibuprofen | 600 mg PO | 600 mg PO | 400 mg PO | 400 mg PO | 200 mg PO | 200 mg PO |
| | TID | TID | TID | TID | TID | TID |
| Oxycodone | 5 mg Q4 H | 4 mg Q4 H | 2.5 mg Q6 H | 2.5 mg Q6 H | 0 mg | 0 mg |
| | (30 mg QD) | (20 mg QD) | (10 mg QD) | (10 mg QD) | | |
| Cannabinol | — | 300 mg QD | 300 mg QD | 300 mg QD | 300 mg QD | 300 mg QD |
| (CBD) | | divided TID | divided TID | divided TID | divided TID | divided TID |

TABLE 4
CBD-Only Facilitated Taper for Moderately Opioid-Dependent Patient

| | Oral Dosing Medication Taper Sample Schedule | | | | | |
|---|---|---|---|---|---|---|
| | Week 1 | Week 2 | Week 3 | Week 4 | Week 5 | Week 6 |
| Acetaminophen | 3000 mg QD | 3000 mg QD | 3000 mg QD | 2000 mg QD | 2000 mg QD | 2000 mg QD |
| | divided TID | divided TID | divided TID | divided TID | divided TID | divided TID |
| Ibuprofen | 600 mg PO | 600 mg PO | 400 mg PO | 400 mg PO | 200 mg PO | 200 mg PO |
| | TID | TID | TID | TID | TID | TID |
| Oxycodone | 10 mg Q4 H | 7.5 mg Q4 H | 5 mg Q4 H | 2.5 mg Q4 H | 2.5 mg Q6 H | 0 mg |
| | (60 mg QD) | (45 mg QD) | (30 mg QD) | (15 mg QD) | (10 mg QD) | |
| Cannabinol | — | 300 mg QD | 300 mg QD | 300 mg QD | 300 mg QD | 300 mg QD |
| (CBD) | | divided TID | divided TID | divided TID | divided TID | divided TID |

### Example 2. CBD- and THC-Facilitated Taper

This example demonstrates that CBD and THC are used to facilitate taper of opioid (oxycodone) use in moderately opioid-dependent patients. For dosing schedules, the following abbreviations are used: BID=2 times per day, every 12 hours; PO=by mouth; QD=daily; Q4H=every 4 hours; Q6H=every 6 hours; TID=3 times per day, every 8 hours.

Table 5 is an example of dosing schedules for illustration purposes only. Depending on the conditions and responses of individual patients, the dosing schedules can be adjusted accordingly.

TABLE 5
CBD- and THC-Facilitated Taper for Moderately Opioid-Dependent Patient

| | Oral Dosing Medication Taper Sample Schedule | | | | | |
|---|---|---|---|---|---|---|
| | Week 1 | Week 2 | Week 3 | Week 4 | Week 5 | Week 6 |
| Acetaminophen | 3000 mg QD | 3000 mg QD | 3000 mg QD | 2000 mg QD | 2000 mg QD | 2000 mg QD |
| | divided TID | divided TID | divided TID | divided TID | divided TID | divided TID |
| Ibuprofen | 600 mg PO | 600 mg PO | 400 mg PO | 400 mg PO | 200 mg PO | 200 mg PO |
| | TID | TID | TID | TID | TID | TID |
| Oxycodone | 10 mg Q4 H | 7.5 mg Q4 H | 5 mg Q4 H | 2.5 mg Q4 H | 2.5 mg Q6 H | 0 mg |
| | (60 mg QD) | (45 mg QD) | (30 mg QD) | (15 mg QD) | (10 mg QD) | |
| Cannabinol | — | 200 mg PO | 200 mg PO | 200 mg PO | 200 mg PO | 200 mg PO |
| (CBD) | | QD | QD | QD | QD | QD |
| Delta-9 | — | 20 mg PO | 20 mg PO | 10 mg PO | 10 mg PO | 5 mg PO |
| Tetrahydrocannabinol | | BID | BID | BID | BID | |
| (THC) | | | | | | |

### Example 3. Psilocybin-Facilitated Dependence Awareness & Taper

This example demonstrates that psilocybin is used to facilitate taper of opioid (oxycodone) use in patients. For dosing schedules, the following abbreviations are used: BID=2 times per day, every 12 hours; PO=by mouth; QD=daily; Q4H=every 4 hours; Q6H=every 6 hours; TID=3 times per day, every 8 hours.

Table 6 is an example of dosing schedules for illustration purposes only. Depending on the conditions and responses of individual patients, the dosing schedules can be adjusted accordingly.

TABLE 6
Psilocybin-Facilitated Dependence Awareness & Taper

| | Oral Dosing Medication Taper Sample Schedule | | | | | |
|---|---|---|---|---|---|---|
| | Week 1 | Week 2 | Week 3 | Week 4 | Week 5 | Week 6 |
| Acetaminophen | 3000 mg QD | 3000 mg QD | 3000 mg QD | 2000 mg QD | 2000 mg QD | 2000 mg QD |
| | divided TID | divided TID | divided TID | divided TID | divided TID | divided TID |
| Ibuprofen | 600 mg PO | 600 mg PO | 400 mg PO | 400mg PO | 200 mg PO | 200 mg PO |
| | TID | TID | TID | TID | TID | TID |
| Oxycodone | 10 mg Q4 H | 7.5 mg Q4 H | 5mg Q4 H | 2.5 mg Q4 H | 2.5 mg Q6 H | 0 mg |
| | (60 mg QD) | (45 mg QD) | (30 mg QD) | (15 mg QD) | (10 mg QD) | |
| Psilocybin (or | 0 mg | 0 mg | 20-30 mg PO | 0 mg | 0 mg | 20-30 mg PO |
| pharmaceutical | | | (once, under | | | (once, under |
| equivalent) | | | supervision) | | | supervision) |

### Example 4. Ketamine-Facilitated Dependence Awareness & Taper

This example demonstrates that ketamine is used to facilitate taper of opioid (oxycodone) use in patients. For dosing schedules, the following abbreviations are used: BID=2 times per day, every 12 hours; PO=by mouth; QD=daily; Q4H=every 4 hours; Q6H=every 6 hours; TID=3 times per day, every 8 hours.

Table 7 is an example of dosing schedules for illustration purposes only. Depending on the conditions and responses of individual patients, the dosing schedules can be adjusted accordingly.

TABLE 7
Ketamine-Facilitated Dependence Awareness & Taper

| | Oral Dosing Medication Taper Sample Schedule | | | | | |
|---|---|---|---|---|---|---|
| | Week 1 | Week 2 | Week 3 | Week 4 | Week 5 | Week 6 |
| Acetaminophen | 3000 mg QD | 3000 mg QD | 3000 mg QD | 2000 mg QD | 2000 mg QD | 2000 mg QD |
| | divided TID | divided TID | divided TID | divided TID | divided TID | divided TID |
| Ibuprofen | 600 mg PO | 600 mg PO | 400 mg PO | 400 mg PO | 200 mg PO | 200 mg PO |
| | TID | TID | TID | TID | TID | TID |
| Oxycodone | 10 mg Q4 H | 7.5 mg Q4 H | 5 mg Q4 H | 2.5 mg Q4 H | 2.5 mg Q6 H | 0 mg |
| | (60 mg QD) | (45 mg QD) | (30 mg QD) | (15 mg QD) | (10 mg QD) | |
| Ketamine | — | — | 1.0-2.0 mg/lb. | — | — | 1.0-2.0 mg/lb. |
| | | | body weight PO | | | body weight PO |
| | | | (once, under | | | (once, under |
| | | | supervision) | | | supervision) |

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermediate components. Likewise, any two components so associated can also be viewed as being “operably connected,” or “operably coupled,” to each other to achieve the desired functionality, and any two components capable of being so associated can also be viewed as being “operably couplable” to each other to achieve the desired functionality. Specific examples of operably couplable include, but are not limited to, physically mate-able and/or physically interacting components and/or wirelessly interactable and/or wirelessly interacting components and/or logically interacting and/or logically interactable components.

The ranges disclosed herein also encompass any and all overlap, sub-ranges, and combinations thereof. Language such as “up to,” “at least,” “greater than,” “less than,” “between,” or the like includes the number recited. Numbers preceded by a term such as “approximately,” “about,” and “substantially” as used herein include the recited numbers (e.g., about 10%=10%), and also represent an amount close to the stated amount that still performs a desired function or achieves a desired result. For example, the terms “approximately,” “about,” and “substantially” may refer to an amount that is within less than 10% of, within less than 5% of, within less than 1% of, within less than 0.1% of, and within less than 0.01% of the stated amount.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting.

The following examples are intended to illustrate various embodiments of the invention. As such, the specific embodiments discussed are not to be construed as limitations on the scope of the invention. It will be apparent to one skilled in the art that various equivalents, changes, and modifications may be made without departing from the scope of the invention, and it is understood that such equivalent embodiments are to be included herein.

1. A method for reducing opioids in a pain-modulating regimen of a patient, the method comprising:
generating, by a computer system, a dosage scheme for the patient based on one or more parameters associated with a health condition of the patient, wherein:
the dosage scheme includes a first dosage scheme for an opioid compound and a first dosage scheme for a cannabinoid compound to be administered to the patient concurrently over a period of time; and
the first dosage scheme for the opioid compound includes a first plurality of dosages with a decreasing amount of an active ingredient in the opioid compound over the period of time;
determining an addiction risk score for the patient based on the dosage scheme;
determining, by the computer system, whether to adjust the dosage scheme based on the addiction risk score for the patient;
in response to determining to adjust the dosage scheme, generating, by the computer system, an adjusted dosage scheme by:
receiving pain data of the patient associated with the pain-modulating regimen;
using at least one trained customizable cannabinoid dosing machine learning model to:
analyze the received pain data,
identify, based on the analysis of the received pain data, one or more patient-specific correlations between opioid dosing of the first dosage scheme for the opioid compound and cannabinoid dosing of the first dosage scheme for the cannabinoid compound, and
generate the adjusted dosage scheme based on the identified one or more patient-specific correlations to alleviate pain of the patient and the addiction risk score, wherein:
the adjusted dosage scheme includes a second dosage scheme for the opioid compound that is different from the first dosage scheme for the opioid compound to be administered to the patient; and
communicating, by the computer system to a drug administration device, instructions for providing the opioid compound in accordance with the adjusted dosage scheme, wherein:
the drug administration device provides physical access to the opioid compound stored in compartments of the drug administration device to the patient to control a quantity, volume, and/or concentration of the opioid compound accessible to the patient.

2. The method of claim 1, further including:
receiving, by the computer system, pain level indicators associated with the patient, wherein the pain level indicators include:
a first pain level indicator based on an input from the patient; and
a second pain level indicator based on biometric data obtained by a wearable device configured to be worn by the patient, and
determining, by the computer system, whether to adjust at least one of the dosage scheme or the adjusted dosage scheme based on the pain level indicators.

3. The method of claim 2, further comprising:
determining, based on the received pain level indicators, at least one relationship between pain-reduction efficacy of the cannabinoid compound and pain-reduction efficacy of the opioid compound,
wherein the adjusted dosage scheme is generated based on the determined at least one relationship and has a predicted pain score that less than a maximum pain score.

4. The method of claim 2, further including:
in response to receiving the pain level indicators associated with the patient, transmitting, by the computer system to a user device associated with the patient, instructions to administer the opioid compound and the cannabinoid compound to the patient in accordance with the adjusted dosage scheme.

5. The method of claim 1, wherein the first dosage scheme for the cannabinoid compound includes a second plurality of dosages with an increasing amount of an active ingredient in the cannabinoid compound over the period of time.

6. The method of claim 1, wherein the dosage scheme further includes a third dosage scheme for a pain relief medication to be administered to the patient concurrently.

7. The method of claim 2, wherein the biometric data obtained by the wearable device includes one or more of: a heart rate, a temperature, a blood pressure, a respiration rate, an oxygenation level, a motor activity level, a pupil constriction/dilation level, and sleep phase information.

8. The method of claim 1, further including:
administering, to the patient, a first set of dosages of the opioid compound and a first set of dosages of the cannabinoid compound in accordance with the dosage scheme; and
administering, to the patient, a second set of dosages of the opioid compound and a second set of dosages of the cannabinoid compound in accordance with the adjusted dosage scheme.

9. The method of claim 4, wherein the instructions include times, amounts, types, and methods of administering the opioid compound and the cannabinoid compound to the patient.

10. The method of claim 1, wherein the opioid compound and the cannabinoid compound are independently administered as pills, capsules, tablets, transdermal patches, injections, tinctures, smokable herbs, or as an inhalable vapor.

11. The method of claim 1, wherein the dosage scheme and the adjusted dosage scheme are configured to gradually reduce the opioid compound from the pain-modulating regimen of the patient by replacing the opioids with the cannabinoid compound.

12. The method of claim 1, further including:
determining, by the computer system, whether an amount of opioid administered to the patient in accordance with the dosage scheme or the adjusted dosage scheme is above a threshold limit; and
reducing the amount of opioid administered to the patient in an instance where the amount of opioid is above the threshold limit.

13. The method of claim 1, further including:
generating the adjusted dosage scheme partially based on an input received from a user of the computer system;
determining, by the computer system, whether an amount of opioid administered to the patient in accordance with the adjusted dosage scheme is above a threshold limit; and
in response to determining that the amount of opioid administered to the patient in accordance with the adjusted dosage scheme is above the threshold limit,
providing, by the computer system, an threshold exceeded indication to the user.

14. A system eliminating opioids from a pain-modulating regimen of a patient, the system comprising:
a drug administration device; and
a computer device programmed to be in communication with the drug administration device, a user device and a wearable device associated with the patient, the computer device configured to:
generate a dosage scheme for the patient based on parameters associated with a health condition of the patient, wherein:
the dosage scheme includes a first dosage scheme for an opioid compound and a first dosage scheme for a cannabinoid compound to be administered to the patient concurrently over a period of time; and
the first dosage scheme for the opioid compound includes a first plurality of dosages with a decreasing amount of an active ingredient in the opioid compound over the period of time;
determine an addiction risk score for the patient based on the dosage scheme;
determine, by the computer, whether to adjust the dosage scheme based on the addiction risk score for the patient; and
generate an adjusted dosage scheme by:
receiving pain data of the patient associated with the pain-modulating regimen; and
using at least one trained customizable cannabinoid dosing machine learning model to:
analyze the received pain data,
identify, based on the analysis of the received pain data, one or more patient-specific correlations between opioid dosing of the first dosage scheme for the opioid compound and cannabinoid dosing of the first dosage scheme for the cannabinoid compound, and
generate the adjusted dosage scheme based on the identified one or more patient-specific correlations to alleviate pain of the patient and the addiction risk score, wherein:
the adjusted dosage scheme includes a second dosage scheme for the opioid compound that is different from the first dosage scheme for the opioid compound to be administered to the patient; and
communicating, by the computer system to a drug administration device, instructions for providing the opioid compound in accordance with the adjusted dosage scheme, wherein:
the drug administration device provides physical access to the opioid compound stored in compartments of the drug administration device to the patient to control a quantity, volume, and/or concentration of the opioid compound accessible to the patient.

15. The system of claim 14, wherein the computer device is further configured to:
receive pain level indicators associated with the patient, wherein the pain level indicators include:
a first pain level indicator that is based on an input from the patient and is received from the user device; and
a second pain level indicator that is based on biometric data obtained from the patient by the wearable device and is received from the wearable device; and
generate the adjusted dosage scheme further based on the first and second pain level indicators.

16. The system of claim 14, wherein the user device is configured to:
receive instructions to administer the opioid compound and the cannabinoid compound to the patient in accordance with the dosage scheme and the adjusted dosage scheme; and
display the instructions to the patient.

17. The system of claim 14, wherein the wearable device is configured to:
obtain biometric data from the patient; and
transmit the biometric data to the computer device thereby enabling the computer device to generate the adjusted dosage scheme.

18. The system of claim 17, wherein:
obtaining the biometric data includes detecting, by one or more sensors of the wearable device, one or more of: a heart rate, a temperature, a blood pressure, a respiration rate, an oxygenation level, a motor activity level, a pupil constriction/dilation level, and sleep phase information of the patient.

19. The system of claim 14, wherein the wearable device is a smartwatch, a wristband, a fitness tracker, or a wearable medical device.

20. A method for administrating drugs to a patient, the method comprising:
generating, by a computer system, an initial dosage scheme for the patient based on parameters associated with a health condition of the patient, wherein:
the initial dosage scheme includes a first dosage scheme for a first drug and a first dosage scheme for a second drug to be administered to the patient concurrently over a period of time; and
the first dosage scheme for the first drug includes a first plurality of dosages with a decreasing amount of an active ingredient in the first drug over the period of time;
receiving, by the computer system, one or more health condition indicators associated with the patient, wherein the one or more health condition indicators include an indicator based on an input from the patient;
generating, by the computer system, a subsequent dosage scheme by:
receiving pain data of the patient; and
using at least one trained customizable machine learning model to:
analyze the received pain data,
identify, based on the analysis of the received pain data, one or more patient-specific correlations between first drug dosing of the first dosage scheme for the first drug and second drug dosing of the first dosage scheme for the second drug, and
generate the subsequent dosage scheme based on the identified one or more patient-specific correlations to alleviate pain of the patient and the one or more health condition indicators; and
communicating, by the computer system to a drug administration device, instructions for providing the opioid compound in accordance with the subsequent dosage scheme, wherein:
the drug administration device provides physical access to the first drug stored in compartments of the drug administration device to the patient to control a quantity, volume, and/or concentration of the first drug accessible to the patient.

21-38. (canceled)

39. The method of claim 1, further comprising:
adjusting parameters of the at least one trained customizable cannabinoid dosing machine learning model to produce a user-selected customized result, wherein
the customized result is selected from at least one of target alleviation of pain, target types of compound formulations, or target speed of decreasing the active ingredient in the opioid compound.

40. The method of claim 1, wherein generating the adjusted dosage scheme further comprises:
receiving a threshold addiction risk score set by a physician or a healthcare provider associated with the patient; and
training the at least one trained customizable cannabinoid dosing machine learning model to maintain the addiction risk score for the patient below the threshold addiction risk score.
